(19) United States
(12) Patent Application Publication
(10) Pub. No.: US 2005/0288279 A1
(43) Pub. Date: Dec. 29, 2005

(54) Phenothiazine derivatives and their method of use

(76) Inventors: Karl K. Rozman, Shawnee Mission, KS (US); Kristian Fried, Idstein (DE); Paul F. Terranova, Overland Park, KS (US); Gunda I. Georg, Lawrence, KS (US); Apurba Dutta, Lawrence, KS (US)

Correspondence Address: STINSON MORRISON HECKER LLP;ATTN: PATENT GROUP, 1201 WALNUT STREET, SUITE 2800, KANSAS CITY, MO, 64106-2150, US

(21) Appl. No.: 10/875,864

(22) Filed: Jun. 24, 2004

(51) Int. Cl. C07D 279/18; A61K 31/5415

(52) U.S. Cl. 514/224.8; 544/39

(57) ABSTRACT

Novel phenothiazine derivatives and their use in the treatment of diabetes mellitus (type I and type II), and as an ovulation inhibitor (contraceptive), cancer chemotherapeutic and/or prophylactic agent, anti-obesity drug (body weight regulator), and immunostimulant.

### CROSS-REFERENCE TO RELATED APPLICATIONS

None.

### STATEMENT REGARDING FEDERALLY SPONSORED RESEARCH OR DEVELOPMENT

None.

### BACKGROUND OF THE INVENTION

A. Polychlorinated Dibenzo-p-Dioxins

Polychlorinated dibenzo-p-dioxins (“PCDDs”), also commonly referred to as “dioxins,” are among the most toxic xenobiotics known. They rank high in the awareness of both the general public and regulatory agencies world-wide. They are ubiquitous compounds of high environmental and biological persistence. Dioxins are subject to biomagnification and, more importantly, bioaccumulation. Therefore, the exposure of humans, being at the top of the food chain, is inevitable. Their toxicological properties, which depend on the pattern of chlorination, in combination with high lipophilicity and an extremely low rate of biotransformation, make PCDDs to a potential risk for human health and the environment.

PCDDs display different toxic potencies among congeners. The lowest LD₅₀ values were observed for congeners having chlorine substituents in the four lateral positions. The potency of PCDDs relative to each other is expressed by toxic equivalency factor (“TEF”) values, which were established by NATO and WHO. 2,3,7,8-Tetrachlorodibenzo-p-dioxin (TCDD) and 1,2,3,7,8-pentachlorodibenzo-p-dioxin (PeCDD) are the two most toxic members with TEF-values defined as 1 (WHO).

LD₅₀ values TCDD vary across species, with guinea pigs being the most sensitive animals (LD₅₀=2.1 μg/kg (Schwetz et al. 1973)) and hamsters being the most resistant (LD₅₀=1157−5051 μg/kg (Eisler 1986)). TCDD has an LD₅₀ of 22.0 μg/kg in male and 45.0 μg/kg in female rats (Schwetz et al. 1973). There is also a very pronounced inter-strain variability as shown by about a 300-fold difference (10 μg/kg and >3000 μg/kg, respectively) between Long-Evans and Han Wistar rats (Pohjanvirta et al. 1990; Pohjanvirta et al. 1993).

PCDDs' detrimental effects are widely known and quite diverse. They include a wasting syndrome (Harris et al. 1973; Seefeld et al. 1984a), carcinogenicity (Luster et al. 1990), endocrine effects on ovulation (Li et al. 1995a; Li et al. 1995b), inhibition of 17-β estradiol-induced uterotrophy (Gallo et al. 1986; Romkes et al. 1989), thymic atrophy (Gupta et al. 1973), and immunosuppression (Miller et al. 1977). In humans, chloracne is the most common symptom of elevated TCDD body burdens. In susceptible individuals, symptoms may occur at TCDD concentrations of 800 ppt based on serum lipid content, although differential diagnosis of acne-like skin conditions is notoriously difficult in adolescents. Most individuals do not show signs below 11,000 ppt (Williams & Wilkins 1992), which corresponds to doses of about 100 μg/kg TCDD (Young 1984). Investigations after accidental exposure also link TCDD to increased risk of digestive tract and respiratory tract cancer, the occurrence of which was elevated in smokers (Ott & Zober 1996). Liver cancer was unequivocally demonstrated in animals only (Kociba et al. 1978; Huff 1992). Out of all the effects shown to occur in animals and humans (Skene et al. 1989) the number one concern for human health is carcinogenicity (Fingerhut et al. 1991). Other effects claimed to occur in humans include hyperkeratinosis, hyperpigmentation, hirsutism, liver damage, elevated blood fat content and cholesterol, intestinal effects with diarrhea, cardiovascular effects, headache, peripheral neuropathy, reduced sensory performance, loss of libido, and psychiatric changes (Abel 1987). Some of these effects are also age-related and therefore difficult to assess if indeed dioxins are contributory or not. In animals, PCDDs are also fetotoxic and teratogenic (Neubert & Meister 1987).

B. Phenothiazines

Phenothiazines are similar in structure to dioxins but have a large number of medical and other uses. Phenothiazines have been used as textile dyes for centuries, especially for silk. Today, phenothiazines are a class of compounds with a multitude of uses. Without any doubt, the most important use lies in medicine. Their application ranges from the most prominent area of antipsychotics/neuroleptics (Shen 1999) and sedatives, anti-histamines and anti-emetics to the treatment of migraine (Stiell et al. 1991), diabetes (Pandey & Pathak 1999), cardiac irregularities (Fauchier et al. 1991; Vizir et al. 1991) and many more.

Unexpected death is a serious side effect of sedative phenothiazines (Mehtonen et al. 1991). Studies on piglets revealed a decrease in the spontaneous occurrence of swallowing, and an increased occurrence of sleep apnea. Thus, the effectiveness of protection mechanisms for the trachea during sleep is decreased (McKelvey et al. 1999).

Most toxic effects are related to exaggerated pharmacological responses. The most troublesome side-effects are related to the extra-pyramidal system. A detailed accounting of them may be found in Goodman & Gilman (Goodman & Gilman 2001).

Classical phenothiazines offer open sites on their aryl rings for the oxidative phase I enzymes of the class cytochrome P450. Ring-hydroxylated metabolites are common and have intrinsic pharmacological effects themselves (Nowak et al. 1990). The phenothiazines' N-substituents possibly offer more sites for metabolic interactions in addition to the common cleavage of the N—R bond (Choo et al. 1990). The formation of the sulfoxide was studied in Aspergillus niger (Parshikov et al. 1999) and is a likely metabolic pathway in higher organisms, as a product of cytochrome P450 activities.

The pharmacological effects of phenothiazines are thought to be directed by different N-substituents. Aliphatic N-substituents (derivatives promazin, chlorpromazine and promethazin) show strong sedative effects. Piperidyl N-substituents (the pecazin-types, e.g. pericazin, thioridazin) have a medium sedative potency. Piperazinyl N-substituents (members of the perphenazin-class) have only weak sedative properties but act as antihistamines, and are strongly anti-psychotic and anti-emetic.

The vast majority of phenothiazines have only been developed for the effects of their N-substituents. Only a handful of studies have been found that investigated the effects of ring-substituents (Jovanovic & Biehl 1987). That study was a comparison between chlorpromazine and 2-methoxypromazine. The latter proved to have no effects compared to its prominent analogue's properties. A likely explanation evokes differences in pharmacokinetics. Due to rapid de-methylation, the half-life of the 2-methoxy-analogue is greatly shortened.

Several alkoxylated phenothiazine dertivatives have also been proposed. See Quelet et al., “Synthesis of 2,3-dimethoxy-10-methylphenothiazines,” Fac. Sci., Paris, Compt Rend. (1964), 258(13); Japanese Patent No. 69-79103 to Ichihara et al. entititled “Methoxy-substituted 10-bromoacetylphenothiazines” (1973); Japanese Patent No. JP-70-6422 to Ichihara et al. entitled “Alkoxy-substituted 10-iodoacetylphenothiazines” (1973); all of which are incorporated by reference. In addition, a few researchers have synthesized several halogenated derivatives of phenothiazines. See Ma et al., “Fluoroescence Study on Phenothiazine Halogenate Derivatives,” Test and Analysis Center Shanzi University, Guangpuzue Yu Guangpu Fenxi, 19(2), 250-252 (1999) (3-bromo-N-ethyl-phenothiazine); Nodiff et al., “Synthesis of Phenothiazines”, Temple University,” Journal of Organic Chemistry, 26, 824-28 (1961); Kumar et al, “Synthesis of 1- and 3-chlorophenothiazines”, University of Rajasthan, Heterocyclic Communications, 8(5), 447-450 (2002); Sharma et al., “Synthesis of Phenothiazines via Smiles Rearrangement,” University of Rajasthan, Heterocylic Communications, 8(2), 195-198 (2002) all of which are incorporated by reference. In addition, Li (1988) and Huang (1997) reported the synthesis of a tetra-substituted derivative in “Synthesis of N-ethylphenothiazine and its derivatives” and “Fluorescence spectra study on 2,3,7,8-tetrachloro-N-ethyl-phenothiazine”, respectively, both of which are incorporated by reference. However, further studies using the synthesis techniques described in these latter Chinese publications indicate that the compound synthesized was actually 1,3,7,9-tetrachloro-N-ethyl-phenothiazine.

The present invention is directed to novel phenothiazine derivatives and their use in the treatment of diabetes mellitus (type I and type II), ovulation inhibitor (contraceptive), cancer chemotherapeutic agent, anti-obesity drug (body weight regulator), immunostimulant, and life-prolonging drug.

### BRIEF SUMMARY OF THE INVENTION

An object of the present invention is to provide novel phenothiazine derivatives.

In accordance with the present invention, phenothiazines substituted with three or four halogens or trihalomethyl groups at the 2,3,7, and 8 positions are provided.

In one aspect of the present invention, phenothiazine compounds having the following formula are provided: 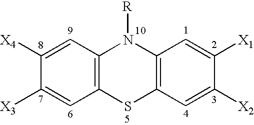
wherein X₁, X₂, X₃, and X₄ are independently hydrogen, halogen, or trihalomethyl, and not more than one of X₁, X₂, X₃, and X₄ is hydrogen; and
wherein R is H or lower alkyl; and
wherein the sulfur is optionally oxidized to sulfoxide or sulfone.

In another aspect, one of X₁, X₂, X₃, and X₄ is halogen; and at least two of X₁, X₂, X₃, and X₄ are independently trihalomethyl; and R is H or lower alkyl; and the sulfur is optionally oxidized.

In still another aspect, two of X₁, X₂, X₃, and X₄ are independently halogen; and at least one of X₁, X₂, X₃, and X₄ is independently trihalomethyl; and R is H or lower alkyl group; and the sulfur is optionally oxidized.

In another aspect of the present invention, at least three of X₁, X₂, X₃, and X₄ are independently halogen or trifluoromethyl; and R is H or lower alkyl; and the sulfur is optionally oxidized.

In a further aspect, three of X₁, X₂, X₃, and X₄, are independently F or trifluoromethyl; and R is lower alkyl.

In yet another aspect, at least three of X₁, X₂, X₃ and X₄, are independently Cl or trifluoromethyl; and R is lower alkyl.

In another aspect, at least three of X₁, X₂, X₃, and X₄ are halogen; and R is H or lower alkyl; and the sulfur is optionally oxidized.

In still another aspect, X₁, and X₂ are both Cl; and at least one of X₃, and X₄ is independently halogen or trifluoromethyl; and R is H or lower alkyl; and the sulfur is optionally oxidized.

In a further aspect, X₁, and X₃ are both Cl; and at least one of X₂, and X₄ is independently halogen or trifluoromethyl; and R is H or lower alkyl; and the sulfur is optionally oxidized.

In a further aspect, X₁, and X₄ are both Cl; and at least one of X₂, and X₃ is independently halogen or trifluoromethyl; and R is H or lower alkyl; and the sulfur is optionally oxidized.

In another aspect, X₂, and X₃ are both Cl; and at least one of X₁, and X₄ are halogen or trifluoromethyl; and R is H or lower alkyl; and the sulfur is optionally oxidized.

In still another aspect, X₁, and X₂, are both F; and at least one of X₃, and X₄ is independently halogen or trifluoromethyl; and R is H or lower alkyl; and the sulfur is optionally oxidized.

In a further aspect, X₁, and X₂ are both I; and least one of X₂, and X₄ is independently halogen or trifluoromethyl; and R is H or lower alkyl; and the sulfur is optionally oxidized.

In still a further aspect, X₁, and X₂ are both Br; and at least one of X₂, and X₄ is independently halogen or trifluoromethyl; and R is H or lower alkyl; and the sulfur is optionally oxidized.

In yet another aspect, X₁ is Cl and X₃ is Br; and at least one of X₂, and X₄ is independently halogen or trifluoromethyl; and R is H or lower alkyl; and the sulfur is optionally oxidized.

In still another aspect, X₁, X₂, and X₃, are all Cl; and at X₄ is hydrogen, halogen, or trifluoromethyl; and R is H or lower alkyl; and the sulfur is optionally oxidized.

In still another aspect, the phenothiazine compound is selected from the group consisting of 2,3,7-trichlorophenothiazine; 2,3,7-trichlorophenothiazine-5-oxide; 2,3,7-trichoropheonothiazine-5,5-dioxide; N-methyl-2,3,7-trichorophenothiazine; N-methyl-2,3,7-trichorophenothiazine-5-oxide; N-methyl-2,3,7-trichorophenothiazine-5,5-dioxide; 2,3,8-trichlorophenothiazine; 2,3,8-trichlorophenothiazine-5-oxide; and 2,3,8-trichoropheonothiazine-5,5-dioxide; N-methyl-2,3,8-trichorophenothiazine; N-methyl-2,3,8-trichorophenothiazine-5-oxide; N-methyl-2,3,8-trichorophenothiazine-5,5-dioxide.

In another aspect, X₁, X₂, and X₃, are all F, and X₄ is hydrogen, halogen, or trifluoromethyl.

In still another aspect, the phenothiazine compound is selected from the group consisting of 2,3,7-trifluorophenothiazine; N-methyl-2,3,7-trifluorophenothiazine; N-methyl-2,3,7-trifluorophenothiazine-5-oxide, 2,3,7-trifluorophenothiazine-5,5-dioxide, 2,3,8-trifluorophenothiazine; N-methyl-2,3,8-trifluorophenothiazine; N-methyl-2,3,8-trifluorophenothiazine-5-oxide, 2,3,8-trifluorophenothiazine-5,5-dioxide.

In another aspect, X₁, X₂, and X₃, are all Br, and X₄ is hydrogen, halogen, or trifluoromethyl.

In still another aspect, the phenothiazine compound is selected from the group consisting of 2,3,7-tribromophenothiazine; 2,3,7-tribromophenothiazine-5-oxide; 2,3,7tribromopheonothiazine-5,5-dioxide; N-methyl-2,3,7-tribromophenothiazine; N-methyl-2,3,7-tribromophenothiazine-5-oxide; N-methyl-2,3,7-tribromophenothiazine-5,5-dioxide; 2,3,8-tribromophenothiazine; 2,3,8-tribromophenothiazine-5-oxide; and 2,3,8-tribromopheonothiazine-5,5-dioxide; N-methyl-2,3,8-tribromophenothiazine; N-methyl-2,3,8-tribromophenothiazine-5-oxide; N-methyl-2,3,8-tribromophenothiazine-5,5-dioxide.

In yet another aspect, each of X₁, X₂, X₃, and X₄ are independently halogen or trifluoromethyl; and R is H or lower alkyl; and the sulfur is optionally oxidized.

In another aspect, X₁, X₂, X₃, and X₄ are all Cl; and R is H or lower alkyl; and the sulfur is optionally oxidized.

In another aspect of the present invention, the phenothiazine compound is selected from the group consisting of 2,3,7,8-tetrachlorophenothiazine; 2,3,7,8-tetrachlorophenothiazine-5-oxide; 2,3,7,8-tetrachlorophenothiazine-5,5-dioxide; N-methyl-2,3,7,8-tetrachlorophenothiazine; N-methyl-2,3,7,8-tetrachlorophenothiazine-5-oxide; N-methyl-2,3,7,8-tetrachlorophenothiazine-5,5-dioxide.

In another aspect of the present invention, X₁, X₂, X₃, and X₄ are all F.

In another aspect of the present invention, the phenothiazine compound is selected from the group consisting of 2,3,7,8-tetrafluorophenothiazine; 2,3,7,8-tetrafluorophenothiazine-5-oxide; 2,3,7,8-tetrafluorophenothiazine-5,5-dioxide; N-methyl-2,3,7,8-tetrafluorophenothiazine; N-methyl-2,3,7,8-tetrafluorophenothiazine-5-oxide; and N-methyl-2,3,7,8-tetrafluorophenothiazine-5,5-dioxide.

In another aspect, X₁, X₂, X₃, and X₄ are all Br.

In still another aspect of the present invention, the phenothiazine compound is selected from the group consisting of 2,3,7,8-tetrabromophenothiazine; 2,3,7,8tetrabromophenothiazine-5-oxide; 2,3,7,8-tetrabromophenothiazine-5,5-dioxide; N-methyl-2,3,7,8-tetrabromophenothiazine; N-methyl-2,3,7,8-tetrabromophenothiazine-5-oxide; and N-methyl-2,3,7,8-tetrabromophenothiazine-5,5-dioxide.

In still another aspect, X₁, X₂, X₃, and X₄ are independently Cl or trifluoromethyl.

In yet another aspect, X₁, X₂, X₃, and X₄ are all trifluoromethyl.

Still another object of the present invention is to provide a phenothiazine compound that causes wasting syndrome at a low level of toxicity.

Another object of the present invention is to provide a phenothiazine compound which alters the set-point for body weight.

Another object of the present invention is to provide a compound which can be used in the treatment of obesity

Yet another object of the present invention is to provide a phenothiazine compound which has cancer-inhibiting effects.

Another object is to provide a phenothiazine compound that operates as a contraceptive by inhibiting ovulation.

Another object of the present invention is to provide a drug which is useful in the treatment of type I and type II diabetes mellitus.

Still another object of the present invention is to provide a compound which operates as an immunostimulant, inducing both a cell mediated and humoral response.

In yet another aspect of the present invention, the compounds are useful to prolong life, and act as insulin-like growth factor (IGF-1) inhibitors.

Thus, still another object is to provide a phenothiazine derivative with a substitution pattern structurally similar to the potent dioxin congener TCDD.

As found in the present invention, the compounds of the present invention do show TCDD-like effects, but they are exhibited at a lower level of toxicity. The hallmark of TCDD exposure in laboratory animals, the wasting syndrome, was shown in animals treated with 2,3,7,8-tetrachlorophenothiazine (TCPT); however, the effects after p.o. dosing were shown at dose rates that suggest a peroral potency of TCPT that is by four orders of magnitude lower. Alteration of the set-point for body weight was observed in agreement with reports on TCDD. It will be appreciated that people are influenced by physical ideals suggested by society and media. Conventional weight-loss programs, which are based on conscious or pharmacological food-restriction, mostly result in a bounce-back of the bodyweight to levels prior to dieting once they are terminated. Influencing bodyweight by liposuction or partial gastrectomy are desperate, high-risk, and invasive methods to regain control. TCPT with its lowering effects on the regulation level of bodyweight could represent an attractive alternative on this market.

The inhibiting effects of TCDD on ovulation in rats demonstrate its potential as endocrine disrupter. Studies with TCPT in guinea pigs have not resulted in the same observations; however, no investigations of the effects of TCDD on guinea pigs have been conducted in that field, either. Therefore, an effect of TCPT in other species, such as rats, yet has to be investigated and is still likely. From an inhibition of ovulation by TCPT, a potential application as a “morning after pill” is within the scope of the present invention. TCPT could also be used as an alternative contraceptive, completely inhibiting menstruation as some oral contraceptives do that are already on the market. If combined with anti-cancer properties, TCPT could become the contraceptive of choice for many women.

### BRIEF DESCRIPTION OF THE DRAWINGS

FIG. 1 illustrates the molecular crystal structure of TCPT.

FIG. 2 shows the body weight comparison of control animal, TCPT-treated animal, and pair-fed control of a dosing period: Day 0 through day 8. The body weight before and after treatment underwent linear regression. Formulas of the trend line are shown next to the respective segment.

FIG. 3 illustrates the low-dose effect of TCPT using quantitative in vitro ethoxyresorufin-o-deethylase studies in rat hepatoma cells.

### DETAILED DESCRIPTION OF PREFERRED EMBODIMENT

The term “halogen atom” as used in the definition means fluorine, chlorine, bromine, iodine, etc.

The term “therapeutically effective amount” refers to an amount sufficient to effect treatment when administered to a patient in need of treatment.

The term “treatment” as used herein refers to the treatment of a disease or medical condition in a patient, such as a mammal (particularly a human) which includes:
(a) preventing the disease or medical condition from occurring, i.e., prophylactic treatment of a patient;
(b) ameliorating the disease or medical condition, i.e., eliminating or causing regression of the disease or medical condition in a patient;
(c) suppressing the disease or medical condition, i.e., slowing or arresting the development of the disease or medical condition in a patient; or
(d) alleviating the symptoms of the disease or medical condition in a patient.

A therapeutically effective amount of the compounds of the present invention may be administered to any animal, preferably a mammal (such as apes, cows, horses, pigs, boars, sheep, rodents, goats, dogs, cats, chickens, monkeys, rabbits, ferrets, whales, and dolphins), and more preferably a human.

The following examples further illustrate the present invention in detail but are not to be construed to limit the scope thereof.

### EXAMPLE 1

### Synthesis of 2,3,7,8-Tetrachlorophenothiazine

A first time synthesis of 2,3,7,8-tetrachlorophenothiazine, described below, was successfully conducted. The usual synthetic pathways for phenothiazines proved to be insufficient to overcome the deactivating effects of the four chlorine atoms on ring cyclization. Out of a multitude of attempted synthetic pathways, only an optimized Ullmann coupling yielded the target compound, shown in the last of three reaction steps depicted below.

TCPT was formed in a short time-window and subsequently decomposed 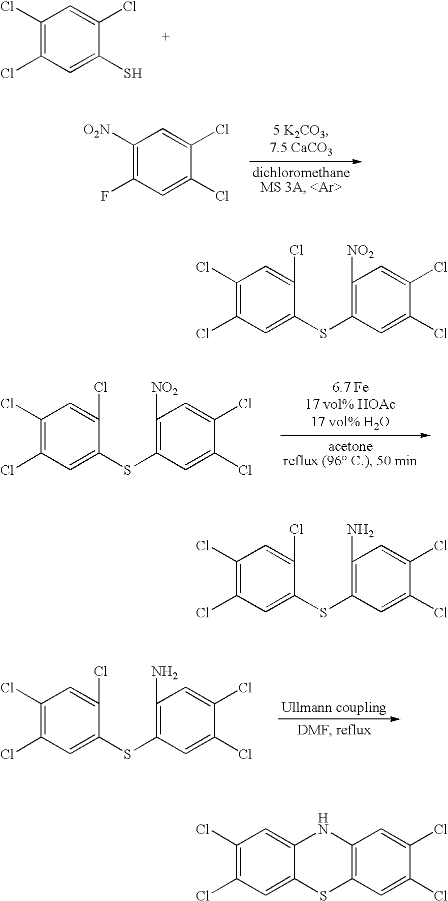
under the rough reaction conditions. At the point of maximum yield (24 h), the reaction was terminated and worked up. The yield of 5.2% after sophisticated purification was low, but enough to generate gram quantities under up-scaled reaction conditions. The first two steps of the synthesis proceeded almost quantitatively once the proper conditions had been worked out.

Details of the three-step procedure for the production of TCPT are set-forth below:

Step 1:

TABLE 1A
Chemicals Used for Step 1

| | | |
|---|---|---|
| 2,4,5- | 1 mole equivalent | Lancaster, Pelham, |
| Trichlorothiophenol, | | NH, USA |
| 97% | | |
| 1,2-Dichloro-4-fluoro-5- | 1 mole equivalent | Aldrich Chemical Co, |
| nitrobenzene, 95% | | Inc., Milwaukee, WI, |
| | | USA |
| Potassium carbonate | 5 mole equivalents | Acros, NJ, USA |
| Calcium carbonate | 7.5 mole equivalents | Fisher Scientific, |
| | | Fair Lawn, NJ, USA |
| Molecular sieves 3 Å | twice the weight of | Aldrich Chemical Co, |
| | (2,4,5- | Inc., Milwaukee, WI, |
| | Trichlorothiophenol + | USA |
| | 1,2-Dichloro-4-fluoro- | |
| | 5-nitrobenzene) | |
| Methylene chloride, | 500 ml per 100 | Fisher Scientific, |
| HPLC grade | mmoles of 2,4,5- | Fair Lawn, NJ, USA |
| | Trichlorothiophenol | |

Preparations:

First, the molecular sieves 3 Å were activated by grinding up with a mortar. The powder was flame-heated under a vacuum and purged with argon gas. The sieves were stored under argon for future use.

Experimental Set-up:

The experimental set-up included an oven-dried round bottom flask (“rbf”) with a stirring magnet and reflux condenser, under argon. The maximum upscaled was 1 L rbf, 500 ml methylene chloride, 100 mmole scale.

Procedure:

The reagents were added into the flask under a stream of argon, with solids added first, and the solvent last. The reflux condenser was set-up and kept under argon throughout the reaction. The rbf was lowered into a pre-heated oil bath at about 55-60° C. and refluxed for 16-21 h. The reaction was checked for completion by thin liquid chromatography (“TLC”) with toluene.

Work-Up:

The mixture was gravity-filtrated through a cellulose filter, collected, and the solid washed with methylene chloride for maximum yield. The solvent was then evaporated, and the mixture was dried on an oil pump over night. The product was a yellow solid.

Purification:

Product was yielded in high purity.

Identification

¹H-NMR (400 MHz, CDCl₃)

d(ppm)=8.41 (1H, H1), 7.83 (1H, H6), 7.75 (1H, H4), 6.79 (1H, H9)

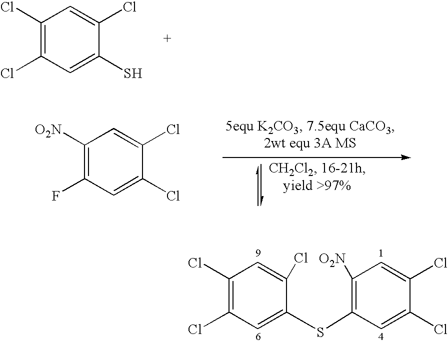

Step 2:

TABLE 1B
Chemicals Used for Step 2

| | | |
|---|---|---|
| KWF1 | 1 mole equivalent | as yielded from Step 1 |
| Iron filings, type | 15 mole equivalents | MCB Manuf. Chem. Inc., |
| IX0240 | | Cincinnati, OH, USA |
| Concentrated or | 0.375 L per 100 | Fisher Scientific, Fair Lawn, |
| glacial acetic acid, | mmoles KWF1 | NJ, USA |
| cert. ACS plus | | |
| Acetone, HPLC | 1.5 L per 100 | Fisher Scientific, Fair Lawn, |
| grade | mmoles KWF1 | NJ, USA |
| Distilled water | 0.375 L per 100 | on site |
| | mmoles KWF1 | |

TABLE 1C
Chemicals used for Preparations, Work-Up and Purification

| | |
|---|---|
| 1N HCl: diluted from conc. | Fisher Scientific, Fair Lawn, NJ, |
| hydrochloric acid, cert. ACS plus | USA |
| Ethanol, absolute 200 proof | Aaper Alc. and Chem. Co, |
| | Shelbyville, KY, USA |
| Diethylether, cert. ACS anhydrous | Fisher Scientific, Fair Lawn, NJ, |
| | USA |
| Celite ® filter agent, 545 | Aldrich Chemical Co, Inc., |
| | Milwaukee, WI, USA |
| Sodium chloride, cert. ACS, crystal | Fisher Scientific, Fair Lawn, NJ, |
| | USA |
| Magnesium sulfate | Fisher Scientific, Fair Lawn, NJ, |
| | USA |
| Hexanes, HPLC grade | Fisher Scientific, Fair Lawn, NJ, |
| | USA |

Preparations:

The iron filings were first activated: They were washed with water until the effluent was almost colorless. Concentrated hydrochloric acid was added and reacted for about a minute. The mixture was washed with distilled water and the surface of the filings was inspected. The procedure was repeated at least three times. After the surface appeared rust-free and metallic, it was washed with HPLC grade ethanol, then with HPLC grade diethyl ether. The mixture was then dried on an oil pump and then stored in an air-tight container for future use.

Experimental Set-up:

The set-up used a three-neck rbf with a mechanical stirrer and reflux condenser. The maximum upscaled was 3 L three-neck rbf, 1.5 L acetone, 0.375 ml concentrated acetic acid, 0.375 ml distilled water, 100 mmole scale.

Procedure

KWF1 was added in acetone in the rbf and heated to reflux. When dissolved, glacial acetic acid and water were added. The yellow suspension was stirred under heat to reflux. Activated iron filings were then added in 30 min intervals (3.5 mole equivalents at a time). The reaction progress was checked by TLC (95% hexanes, 5% ethyl acetate) and more iron was added.

Work-Up

The hot reaction mixture was filtrated through Celite®, most easily on a wide column. The filter agent was washed thoroughly for quantitative yield. The pH-value of the filtrate was adjusted to 8 with sodium hydroxide solution. KWF2 was then extracted from the liquid with diethyl ether. The organic phase was shaken with sodium carbonate solution and brine and then dried over magnesium sulfate before the solvent was evaporated. The crude product was a light-brownish solid.

Purification

The impurities have a higher solubility in hexanes than KWF2 does. Therefore, the crude product was suspended in hexanes at room temperature and stirred for about 1 hour. 250 ml hexanes were used for 15 g crude product. The purified KWF2 was filtrated off and the solid dried under oilpump vacuum. The product was a yellow solid.

Identification

¹H-NMR (400 MHz, CDCl₃)

d(ppm)=7.53 (1H, H₆), 7.48 (1H, H₉), 6.96 (1H, H₄), 6.68 (1H, H₁), 4.36 (2H, NH₂)
Step 2

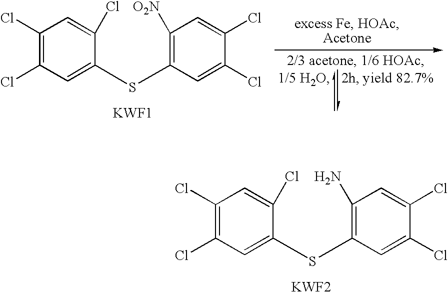

Step 3

TABLE 1D
Chemicals Used for Step 3

| | | |
|---|---|---|
| KWF2 | 1 mole equivalent | as yielded from Step 2 |
| Sodium carbonate | 1 mole equivalent | Fisher Scientific, Fair |
| | | Lawn, NJ, USA |
| Copper iodide, 98% | 0.2 mole equivalents | Acros, NJ, USA |
| Copper | 0.2 mole equivalents | freshly prepared |
| Dimethyl formamide, | 400 ml per 10 mmoles | Aldrich Chemical Co, |
| Sure Seal ® | KWF2 | Inc., Milwaukee, WI, |
| | | USA |

TABLE 1E
Chemicals used for Preparations, Work-Up and Purification

| | |
|---|---|
| Potassium iodide | Lancaster, Pelham, NH, USA |
| Carbon decolorizing, alkaline, Norit A | Fisher Scientific, Fair Lawn, |
| | NJ, USA |
| Celite ® filter agent, 545 | Aldrich Chemical Co, Inc., |
| | Milwaukee, WI, USA |
| Ethanol, absolute 200 proof | Aaper Alc. and Chem. Co, |
| | Shelbyville, KY, USA |
| Diethylether cert. ACS anhydr | Fisher Scientific, Fair Lawn, |
| | NJ, USA |
| Copper sulfate pentahydrate, 98% ACS | Aldrich Chemical Co, Inc., |
| reag. | Milwaukee, WI, USA |
| Zinc powder, 98% | Lancaster, Pelham, NH, USA |
| 1N HCl: diluted from conc. hydrochloric | Fisher Scientific, Fair Lawn, |
| acid, cert. ACS plus | NJ, USA |
| Tetrahydrofuran, certified | Fisher Scientific, Fair Lawn, |
| | NJ, USA |
| Sodium chloride, cert. ACS, crystal | Fisher Scientific, Fair Lawn, |
| | NJ, USA |
| Magnesium sulfate | Fisher Scientific, Fair Lawn, |
| | NJ, USA |
| Hexanes, HPLC grade | Fisher Scientific, Fair Lawn, |
| | NJ, USA |
| tert-butyl methyl ether, 99.8% HPLC | Aldrich Chemical Co, Inc., |
| grade | Milwaukee, WI, USA |

Preparations:

First, clean copper iodide was prepared. A solution of 375 g potassium iodide in 300 ml distilled water was prepared. Next, 60 g copper iodide was added and stirred for 5 minutes at room temperature. Decolorizing charcoal was added and stirring was continued for another 5 minutes. The mixture was filtered through Celite®. About 1.5 L distilled water was added to the liquid. The precipitated copper iodide was collected by vacuum filtration, washed with HPLC grade ethanol and then washed with HPLC grade diethyl ether. The purified copper iodide was dried on a oil pump and protected from light. The material was stored dark and under argon for future use.

Fresh copper was prepared within one hour of usage. About 0.500 g copper sulfate (CuSO₄.5H₂O) and 0.131 g zinc dust were used to prepare fresh copper for a 10 mmole scale. The copper sulfate was dissolved in distilled water and cooled to 0° C. with an ice bath. At 0° C., zinc dust was added. After complete precipitation (i.e., decolorization), the supernatant was decanted, and the precipitate was washed with 1N hydrochloric acid. The mixture was stirred until no generation of hydrogen gas was noted. The mixture was kept at 0° C., decanted, and washed with distilled water until neutral. Next, the mixture was decanted and washed with HPLC grade ethanol and stored under ethanol until shortly before use. Lastly, the mixture was decanted and washed with Sure Seal® DMF and flushed into a reaction vessel.

Experimental Set-up:

To set-up, an oven-dried rbf with a stirring magnet and reflux condenser, under argon was used. The maximum upscaled was 2 L rbf, 1 L dimethyl formamide, 25 mmole scale.

Procedure

All reagents and catalysts were added under a stream of argon. The rbf was lowered into a pre-heated oil bath at about 185° C. and stirred for about 24 hours. A check of the reaction mix by TLC (95% hexanes, 5% ethyl acetate) revealed still existing starting material, but with increasing reaction time, another product was formed and the amount of target compound decreased.

Work-Up

The heat was turned off, and the oil bath was removed. When the rbf was about hand-warm, the mixture was poured into double the volume of ice water under vigorous stirring. The addition of sodium chloride enhanced precipitation considerably. The precipitate was allowed to age and then filtrated by gravity through a cellulose filter (for a 25 mmole scale, a filter paper of 50 cm diameter and a kitchen-drainer proved most practical). The precipitate was washed with distilled water, then vacuum-filtered and dried. The crude product was then dissolved/suspended in tetrahydrofuran (2 L THF for a 35 mmole scale) to extract KWF3 in addition to other products. Because the KWF3 degraded when kept in acetone for longer periods of time, THF was used. The addition of sodium chloride proceeded drying over magnesium sulfate and evaporation of the solvent. The crude product was a black, tar-like goo.

Purification

To purify, column chromatography on silica gel was used with a solvent mixture of 94% hexanes and 6% tert.-butyl methyl ether, and the crude product was put on silica gel before put on the column. A by-product has the same retention factor (R_{f} value) as the target compound KWF3 (TCPT) (˜0.22). After preliminary clean-up on a few columns, the black constituents were removed successfully. The first columns were used to remove KWF2 and other fast-running compounds. The solvent-fractions that followed were saved, trying to cut off only the major fraction of more slowly moving impurities. At the last column, KWF3 was put on the column as a solid powder and until the end mostly stayed accumulated on top of the column and was then flushed down with acetone. The mixture was evaporated immediately since TCPT decomposes in acetone. The product was a white/grayish-purple solid.

Identification

¹H-NMR (400 MHz, CDCl₃)

d(ppm)=7.03 (2H, H1,9), 6.64 (2H, H4,6), 5.83 (1H, NH)

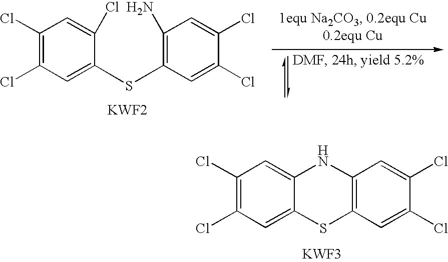

### EXAMPLE 2

### First Crystal Structure of 2,3,7,8-Tetrachlorophenothiazine

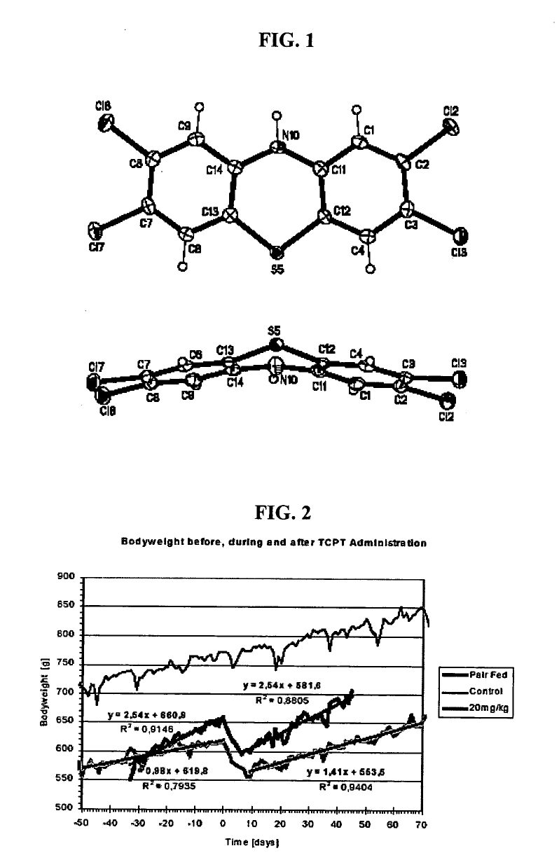

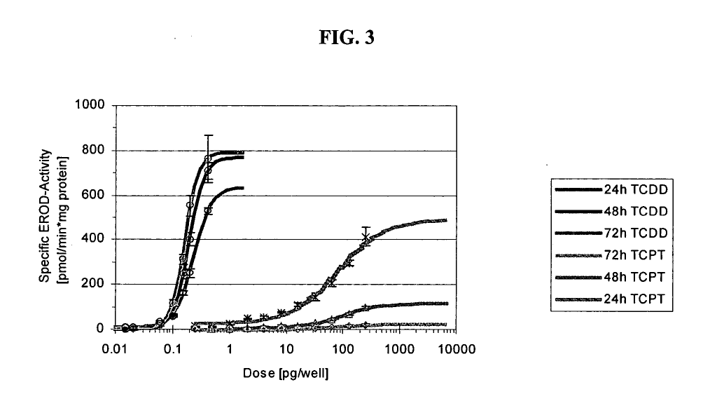

X-ray crystallographic measurements on an orthorhombic crystal resulted in the first crystal structure of TCPT. FIG. 1 illustrates the molecular crystal structure of TCPT from a view perpendicular to the ring system and the bent structure along the heteroatoms. A pink needle-shaped crystal of dimensions 0.41×0.10×0.08 mm was selected for structural analysis. Intensity data for this compound were collected using a Bruker APEX ccd area detector mounted on a Bruker D8 goniometer using with graphite-monochromated Mo Ka radiation (λ=0.71073 Å). See (a) Data Collection: SMART Software Reference Manual (1994). Bruker-AXS, 6300 Enterprise Dr., Madison, Wis. 53719-1173, USA. (b) Data Reduction: SAINT Software Reference Manual (1995). Bruker-AXS, 6300 Enterprise Dr., Madison, Wis. 53719-1173, USA. The sample was cooled to 100(2) K. The intensity data were measured as a series of ω oscillation frames each of 0.25° for 15 sec/frame. The detector was operated in 512×512 mode and was positioned 5.054 cm from the sample. Coverage of unique data was 99.9% complete to 25.99 degrees in θ. Cell parameters were determined from a non-linear least squares fit of 5247 peaks in the range 2.36<θ<26.00°. A total of 6689 data were measured in the range 1.96<θ<25.99°. The data were corrected for absorption by the semi-empirical method giving minimum and maximum transmission factors of 0.6567 and 0.9158. See G. M. Sheldrick (2000). SADABS. Program for Empirical Absorption Correction of Area Detector Data. University of Göttingen, Germany. The data were merged to form a set of 2155 independent data with R(int)=0.0233.

The orthorhombic space group Pna2₁ was determined by systematic absences and statistical tests and verified by subsequent refinement. The structure was solved by direct methods and refined by full-matrix least-squares methods on F². See (a) G. M. Sheldrick (1994). SHELXTL Version 5 Reference Manual. Bruker-AXS, 6300 Enterprise Dr., Madison, Wis. 53719-1173, USA. (b) International Tables for Crystallography, Vol C, Tables 6.1.1.4, 4.2.6.8, and 4.2.4.2, Kluwer: Boston (1995). Hydrogen atom positions were initially determined by geometry and refined by a riding model. Non-hydrogen atoms were refined with anisotropic displacement parameters. Hydrogen atom displacement parameters were set to 1.2 (1.5 for methyl) times the displacement parameters of the bonded atoms. A total of 163 parameters were refined against 1 space group restraint and 2155 data to give wR(F²)=0.0764 and S=1.065 for weights of w=1/[σ² (F²)+(0.0600 P)²], where P=[Fₒ²+2F_{c}²]/3. The final R(F) was 0.0284 for the 2122 observed, [F>4σ(F)], data. The largest shift/s.u. was 0.001 in the final refinement cycle. The final difference map had maxima and minima of 0.521 and −0.225 e/Å³, respectively. The absolute structure was determined by refinement of the Flack parameter. See H. D. Flack, Acta Cryst. A39, 876-881 (1983). The polar axis restraint was taken from Flack and Schwarzenbach. See H. D. Flack and D. Schwarzenbach, Acta Cryst. A44, 499-506 (1988).

The results are shown below in Tables 2A to 2G below

TABLE 2A
Crystal data and structure refinement

| | |
|---|---|
| Empirical formula | C₁₂H₅Cl₄NS |
| Formula weight | 337.03 |
| Crystal system | Orthorhombic |
| Space group | Pna2₁ |
| Unit cell dimensions | a = 20.7356(18) Å α = 90° |
| | b = 15.4686(13) Å β = 90° |
| | c = 3.7921(3) Å γ = 90° |
| Volume | 1216.32(18) Å³ |
| Z, Z′ | 4, 1 |
| Density (calculated) | 1.840 Mg/m³ |
| Wavelength | 0.71073 Å |
| Temperature | 100(2) K |
| F(000) | 672 |
| Absorption coefficient | 1.120 mm⁻¹ |
| Absorption correction | Semi-empirical from equivalents |
| Max. and min. transmission | 0.9158 and 0.6567 |
| Theta range for data collection | 1.96 to 25.99° |
| Reflections collected | 6689 |
| Independent reflections | 2155 [R(int) = 0.0233] |
| Data/restraints/parameters | 2155/1/163 |
| wR(F² all data) | wR2 = 0.0764 |
| R(F obsd data) | R1 = 0.0284 |
| Goodness-of-fit on F² | 1.065 |
| Observed data [I > 2□(I)] | 2122 |
| Absolute structure parameter | 0.01(7) |
| Largest and mean shift/s.u. | 0.001 and 0.000 |
| Largest diff. peak and hole | 0.521 and −0.225 e/Å³ |

wR2 = {Σ [w(Fₒ² − F_{c}²)²]/Σ [w(Fₒ²)²]}^{1/2}
R1 = Σ ||Fₒ| − |F_{c}||/Σ |Fₒ|

TABLE 2B
Atomic coordinates and equivalent isotropic displacement parameters
U(eq) is defined as one third of the trace of the orthogonalized Uᵢⱼ tensor.

| | x | y | z | U(eq) |
|---|---|---|---|---|
| C(1) | 0.57689(11) | 0.77389(15) | 0.5877(7) | 0.0177(5) |
| C(2) | 0.51286(11) | 0.79932(14) | 0.5582(7) | 0.0176(5) |
| Cl(2) | 0.49186(3) | 0.90198(4) | 0.69331(19) | 0.02254(16) |
| C(3) | 0.46771(10) | 0.74260(16) | 0.4228(7) | 0.0166(5) |
| Cl(3) | 0.38742(2) | 0.77147(4) | 0.37295(18) | 0.02273(16) |
| C(4) | 0.48659(10) | 0.66019(15) | 0.3198(7) | 0.0165(5) |
| S(5) | 0.57261(2) | 0.53227(4) | 0.17962(18) | 0.01547(15) |
| C(6) | 0.67314(10) | 0.43390(15) | 0.4049(7) | 0.0156(5) |
| C(7) | 0.73396(10) | 0.42031(15) | 0.5383(7) | 0.0175(5) |
| Cl(7) | 0.76302(3) | 0.31494(4) | 0.56922(17) | 0.02295(17) |
| C(8) | 0.77122(10) | 0.48909(16) | 0.6544(7) | 0.0185(5) |
| Cl(8) | 0.84820(3) | 0.47391(4) | 0.8203(2) | 0.02428(17) |
| C(9) | 0.74672(10) | 0.57235(16) | 0.6416(7) | 0.0186(5) |
| N(10) | 0.66198(8) | 0.67132(13) | 0.4891(7) | 0.0202(5) |
| C(11) | 0.59645(11) | 0.69274(15) | 0.4762(7) | 0.0167(5) |
| C(12) | 0.55039(9) | 0.63482(14) | 0.3440(6) | 0.0142(4) |
| C(13) | 0.64829(10) | 0.51734(14) | 0.3841(7) | 0.0150(5) |
| C(14) | 0.68528(10) | 0.58688(15) | 0.5071(7) | 0.0157(5) |

TABLE 2C
Bond lengths [Å] and angles [°].

| | | | |
|---|---|---|---|
| C(1)—C(11) | 1.385(3) | C(6)—H(6) | 0.9500 |
| C(1)—C(2) | 1.389(3) | C(7)—C(8) | 1.386(3) |
| C(1)—H(1) | 0.9500 | C(7)—Cl(7) | 1.742(2) |
| C(2)—C(3) | 1.382(3) | C(8)—C(9) | 1.385(3) |
| C(2)—Cl(2) | 1.725(2) | C(8)—Cl(8) | 1.732(2) |
| C(3)—C(4) | 1.390(3) | C(9)—C(14) | 1.391(3) |
| C(3)—Cl(3) | 1.734(2) | C(9)—H(9) | 0.9500 |
| C(4)—C(12) | 1.383(3) | N(10)—C(14) | 1.394(3) |
| C(4)—H(4) | 0.9500 | N(10)—C(11) | 1.400(3) |
| S(5)—C(12) | 1.765(2) | N(10)—H(10) | 0.9499 |
| S(5)—C(13) | 1.766(2) | C(11)—C(12) | 1.402(3) |
| C(6)—C(7) | 1.375(3) | C(13)—C(14) | 1.401(3) |
| C(6)—C(13) | 1.392(3) | | |
| C(11)—C(1)—C(2) | 120.8(2) | C(9)—C(8)—Cl(8) | 118.46(18) |
| C(11)—C(1)—H(1) | 119.6 | C(7)—C(8)—Cl(8) | 121.66(18) |
| C(2)—C(1)—H(1) | 119.6 | C(8)—C(9)—C(14) | 119.9(2) |
| C(3)—C(2)—C(1) | 119.8(2) | C(8)—C(9)—H(9) | 120.0 |
| C(3)—C(2)—Cl(2) | 121.60(18) | C(14)—C(9)—H(9) | 120.0 |
| C(1)—C(2)—Cl(2) | 118.56(18) | C(14)—N(10)—C(11) | 124.05(19) |
| C(2)—C(3)—C(4) | 119.7(2) | C(14)—N(10)—H(10) | 109.1 |
| C(2)—C(3)—Cl(3) | 121.83(18) | C(11)—N(10)—H(10) | 116.0 |
| C(4)—C(3)—Cl(3) | 118.43(18) | C(1)—C(11)—N(10) | 119.2(2) |
| C(12)—C(4)—C(3) | 120.7(2) | C(1)—C(11)—C(12) | 119.3(2) |
| C(12)—C(4)—H(4) | 119.6 | N(10)—C(11)—C(12) | 121.5(2) |
| C(3)—C(4)—H(4) | 119.6 | C(4)—C(12)—C(11) | 119.6(2) |
| C(12)—S(5)—C(13) | 101.22(11) | C(4)—C(12)—S(5) | 118.76(17) |
| C(7)—C(6)—C(13) | 120.1(2) | C(11)—C(12)—S(5) | 121.51(16) |
| C(7)—C(6)—H(6) | 119.9 | C(6)—C(13)—C(14) | 119.4(2) |
| C(13)—C(6)—H(6) | 119.9 | C(6)—C(13)—S(5) | 118.38(17) |
| C(6)—C(7)—C(8) | 120.7(2) | C(14)—C(13)—S(5) | 122.16(17) |
| C(6)—C(7)—Cl(7) | 119.02(19) | C(9)—C(14)—N(10) | 119.1(2) |
| C(8)—C(7)—Cl(7) | 120.26(17) | C(9)—C(14)—C(13) | 120.0(2) |
| C(9)—C(8)—C(7) | 119.9(2) | N(10)—C(14)—C(13) | 120.9(2) |

TABLE 2D
Anisotropic displacement parameters (Å² × 10³).
The anisotropic displacement factor exponent takes the form:
−2π²[h²a * ²U₁₁ + . . . + 2hka * b * U₁₂]

| | U₁₁ | U₂₂ | U₃₃ | U₂₃ | U₁₃ | U₁₂ |
|---|---|---|---|---|---|---|
| C(1) | 23(1) | 16(1) | 14(1) | −2(1) | −1(1) | −3(1) |
| C(2) | 28(1) | 11(1) | 14(1) | 2(1) | 3(1) | 3(1) |
| Cl(2) | 31(1) | 14(1) | 23(1) | −2(1) | 1(1) | 4(1) |
| C(3) | 18(1) | 20(1) | 12(1) | 4(1) | 2(1) | 3(1) |
| Cl(3) | 20(1) | 22(1) | 25(1) | 0(1) | −1(1) | 5(1) |
| C(4) | 21(1) | 17(1) | 12(1) | 4(1) | 0(1) | −3(1) |
| S(5) | 19(1) | 14(1) | 14(1) | −3(1) | −(1) | 1(1) |
| C(6) | 19(1) | 16(1) | 11(1) | −1(1) | 5(1) | −3(1) |
| C(7) | 23(1) | 14(1) | 16(1) | 2(1) | 5(1) | 5(1) |
| Cl(7) | 24(1) | 18(1) | 27(1) | 2(1) | 3(1) | 7(1) |
| C(8) | 16(1) | 24(1) | 16(1) | 2(1) | 2(1) | 1(1) |
| Cl(8) | 17(1) | 29(1) | 28(1) | 4(1) | −1(1) | 2(1) |
| C(9) | 19(1) | 21(1) | 16(1) | 1(1) | 0(1) | −4(1) |
| N(10) | 18(1) | 15(1) | 27(1) | −2(1) | −2(1) | −4(1) |
| C(11) | 23(1) | 15(1) | 13(1) | 5(1) | 1(1) | 0(1) |
| C(12) | 21(1) | 12(1) | 10(1) | 3(1) | 2(1) | 2(1) |
| C(13) | 17(1) | 18(1) | 10(1) | 2(1) | 2(1) | −1(1) |
| C(14) | 19(1) | 16(1) | 12(1) | 2(1) | 2(1) | −2(1) |

TABLE 2E
Hydrogen coordinates and isotropic displacement parameters

| | x | y | z | U(eq) |
|---|---|---|---|---|
| H(1) | 0.6076 | 0.8126 | 0.6854 | 0.021 |
| H(4) | 0.4553 | 0.6208 | 0.2317 | 0.020 |
| H(6) | 0.6480 | 0.3862 | 0.3267 | 0.019 |
| H(9) | 0.7719 | 0.6195 | 0.7246 | 0.022 |
| H(10) | 0.6883 | 0.7077 | 0.6305 | 0.024 |

TABLE 2F
Torsion angles [°].

| | |
|---|---|
| C(11)—C(1)—C(2)—C(3) | 1.3(4) |
| C(11)—C(1)—C(2)—Cl(2) | −178.7(2) |
| C(1)—C(2)—C(3)—C(4) | 0.6(4) |
| Cl(2)—C(2)—C(3)—C(4) | −179.46(19) |
| C(1)—C(2)—C(3)—Cl(3) | −178.9(2) |
| Cl(2)—C(2)—C(3)—Cl(3) | 1.1(3) |
| C(2)—C(3)—C(4)—C(12) | −1.4(4) |
| Cl(3)—C(3)—C(4)—C(12) | 178.0(2) |
| C(13)—C(6)—C(7)—C(8) | 0.2(4) |
| C(13)—C(6)—C(7)—Cl(7) | 178.69(19) |
| C(6)—C(7)—C(8)—C(9) | 1.1(4) |
| Cl(7)—C(7)—C(8)—C(9) | −177.5(2) |
| C(6)—C(7)—C(8)—Cl(8) | −179.5(2) |
| Cl(7)—C(7)—C(8)—Cl(8) | 2.0(3) |
| C(7)—C(8)—C(9)—C(14) | −1.1(4) |
| Cl(8)—C(8)—C(9)—C(14) | 179.5(2) |
| C(2)—C(1)—C(11)—N(10) | 176.0(2) |
| C(2)—C(1)—C(11)—C(12) | −2.3(4) |
| C(14)—N(10)—C(11)—C(1) | 156.3(3) |
| C(14)—N(10)—C(11)—C(12) | −25.5(4) |
| C(3)—C(4)—C(12)—C(11) | 0.4(4) |
| C(3)—C(4)—C(12)—S(5) | −175.44(19) |
| C(1)—C(11)—C(12)—C(4) | 1.4(4) |
| N(10)—C(11)—C(12)—C(4) | −176.8(2) |
| C(1)—C(11)—C(12)—S(5) | 177.2(2) |
| N(10)—C(11)—C(12)—S(5) | −1.0(3) |
| C(13)—S(5)—C(12)—C(4) | −163.1(2) |
| C(13)—S(5)—C(12)—C(11) | 21.1(2) |
| C(7)—C(6)—C(13)—C(14) | −1.3(4) |
| C(7)—C(6)—C(13)—S(5) | 175.2(2) |
| C(12)—S(5)—C(13)—C(6) | 161.3(2) |
| C(12)—S(5)—C(13)—C(14) | −22.3(2) |
| C(8)—C(9)—C(14)—N(10) | −178.6(2) |
| C(8)—C(9)—C(14)—C(13) | −0.1(4) |
| C(11)—N(10)—C(14)—C(9) | −157.4(3) |
| C(11)—N(10)—C(14)—C(13) | 24.2(4) |
| C(6)—C(13)—C(14)—C(9) | 1.3(4) |
| S(5)—C(13)—C(14)—C(9) | −175.0(2) |
| C(6)—C(13)—C(14)—N(10) | 179.8(2) |
| S(5)—C(13)—C(14)—N(10) | 3.4(3) |

TABLE 2G
Hydrogen bonds [Åand °]

| | d(D- | | | |
|---|---|---|---|---|
| D-H . . . A | H) | d(H . . . A) | d(D . . . A) | <(DHA) |
| N(10)—H(10) . . . Cl(7)#1 | 0.95 | 2.56 | 3.492(2) | 168.0 |

Symmetry transformations used to generate equivalent atoms:
#1 −x + 3/2, y + 1/2, z + 1/2

The information gained from the crystal structure was important for the structural comparison of TCPT and TCDD. TCDD has a perfectly planar structure (Boer et al. 1972). This example showed that the molecular structure of TCPT is non-planar due to the different heteroatoms positioned in the central ring. Semi-empirical calculations by the present inventor had predicted an angle formed between the two aryl rings of 160.2° for TCPT; 149.2° and 154.3° for its sulfoxide and sulfone, respectively (Fried 2000). The successful X-ray crystallography revealed an angle of 161.5° for TCPT, which supports these calculations. With different stereochemistry, some receptor-interactions of TCPT and TCDD are likely to differ, which in turn could be related to macroscopic manifestations of toxicity.

### EXAMPLE 3

### Acute Toxicity in Adult Male and Female Dunkin-Hartley Guinea Pigs and Adult Female Sprague Dawley Rats

In this example, dose-range finding studies were performed with a small number of animals to accommodate the limited availability of TCPT. Guinea pigs were chosen for the studies because they represent the most sensitive species for dioxin toxicity. Because of the close structural similarity to TCDD, it was assumed that this would be the case also for TCPT. Rats were also investigated as being the standard kinetic model.

1. Guinea Pigs.

Six adult male Dunkin-Hartley guinea pigs were given daily p.o. doses of TCPT. Due to its insolubility, TCPT was administered as a solid in size 9 gelatin capsules and dosing devices purchased from Torpack, Inc. (Fairfield, N.J.). The dosage and results were as follows:

| Animal | Dosage (mg/kg/d) | Result |
|---|---|---|
| 1 | 25 for 18 days | survived |
| 2 | l00 for 8 days | died |
| 3 | 50, 10, 25, 50 (dose- | died |
| | range finding) for | |
| | 17 days | |
| 4-6 | 27.5, 44, 50, 57, 67, | survived |
| | 75 for 13 days | |

The experimental design of daily dosing accounted for the short half-life known from clinically used phenothiazines and the results of prior unpublished studies with 1,2,3,7,8,9-hexachlorophenothiazine. Based on this example, a dose for the acute lethal toxicity of TCPT was broadly calculated to be about p.o. 25-50 mg/kg/day with 8-11 days of dosing until death. The development of tolerance was noticed in survivors.

During ovulation studies (see Example 4 below), an adult female was lost after having been dosed with 10 mg/kg/day for 9 consecutive days; the animal died on day 13. Single intravenous administration of 10 mg/kg TCPT in acetone (0.5 ml/kg) showed no observable effects.

2. Rats.

Adult female Sprague-Dawley rats were injected 5, 10 and 30 mg/kg TCPT in acetone (1 ml/kg). Both animals receiving 30 mg/kg died within five seconds. Animals dosed with 10 mg/kg became unconscious for 30 min, whereas in the 5 mg/kg dose group, the rats lost consciousness for 3 min only, likely representing the effects of acetone in this case. The dose for the acute lethal toxicity of TCPT in rats was therefore broadly calculated to be about i.v. 10-30 mg/kg.

### EXAMPLE 4

### Observations on Death

1. Guinea Pigs.

This example first looked at daily p.o. administration to guinea pigs. Eleven guinea pigs received multiple doses of TCPT, with dosages ranging from 2.5 to 100 mg/kg/day. All animals showed body weight loss. Only females (less than 5 mg/kg/day) males (less than 25 mg/kg/day) with constant doses were able to stabilize their body weight during the dosing period. All guinea pigs that were to die constantly lost bodyweight throughout the whole study up to a point of complete feed refusal and starvation. The death of one animal five days after cessation of dosing indicates the existence of a “point of no return.” Weight loss progressed thereafter. With time, units of feces from dosed animals sporadically contained bubbles which seemed to be intestinal gases contained in a film of solidifying mucus. This observation was made dose-independently and also occurred in one control animal in a single instance. Consultations with pathologists at Bayer Crop Science in Stilwell, KS did not reveal symptoms known from studies with other compounds. Eventually, the animals became diarrhetic. With increasing weight loss, the animals' fur became increasingly puffy. Saliva wetted their feed and their lower neck down to the stomach as feed intake decreased. Dosed animals were often found sitting with their front paws on their feed bowl and digging through their feed than the control animals, giving rise to the assumption that the animals felt hunger but could not act on it. Welcoming squeaks and excited jumping (popcorning) upon arrival of the conductor of the studies were reduced and eventually vanished. Control animals showed these welcoming behaviors throughout the whole study, which leads to the conclusion that the dosing procedure itself had no effect on it. The overall activity of all dosed animals decreased. Purring upon handling endured until death. It is assumed that a general decrease in wellness caused the observed decrease in activity and interactions. The animals became apathetic. About one day before death, mucus was observed on eyes, snout and mouth.

Autopsy revealed the loss of all adipose tissue as expected. The gastrointestinal tract was empty or filled with a brown liquid and was slimy from the outside.

2. Rats.

This example also investigated two rats receiving a single i.v. administration of 30 mg/kg TCPT in acetone (1 ml/kg). The dosed rats first jumped, ran one round in their cage and dropped dead within five seconds after dosing. Those animals that survived lower doses showed breathing from the diaphragm rather than from the chest during their coma. Due to the unexpectedly rapid onset of death, no more detailed observations, e.g. on the heart rate, could be made.

After p.o. administration of various doses during pentobarbital-induced anesthesia, rats breathed very shallow and in long intervals. Survivors (of the lower doses) recovered after two minutes. One death candidate was re-animated by CPR (chest massage and artificial ventilation with a rubber pipette bulb) and seemed to recover but died about 15 minutes later.

### EXAMPLE 5

### Inner-Species Comparison

### LD₅₀ TCPT vs. TCDD in the Guinea Pig and Rat

The toxicity of TCPT was next compared to TCDD using the preliminary studies conducted in Examples 3 and 4. For reasons of simplicity, the dose ranges of acute lethal toxicity found for TCPT is here referred to as LD₅₀.

For guinea pigs (female), the p.o. LD₅₀ of TCPT was estimated to be about 25-50 mg/kg. This is roughly 10,000 times more than the reported p.o. LD₅₀ of 2.1 μg/kg for TCDD (Schwetz et al. 1973).

For rats, the i.v. LD₅₀ of TCPT was estimated to be about 10-30 mg/kg. This is roughly 1,000 times more than the reported i.v. LD₅₀ of TCDD of 25 μg/kg for TCDD (Schwetz et al. 1973).

The difference of one order of magnitude between the i.v. data from rats and the p. o. data from guinea pigs could have several reasons. First, a strong first pass effect could cause such a difference. Second, a lower absorption of TCPT from the gastrointestinal tract as compared to TCDD could also account for the variation. Comparing human data, the absorption of TCDD is >86% (Poiger & Schlatter 1989), whereas that of chlorpromazine ranges between 10.5 and 25.7% (Koytchev et al. 1994). Third, the different doses of acetone vs. gelatin as a carrier may play a role.

### EXAMPLE 6

### Inter-Species Comparison: LD₅₀ TCPT vs. TCDD in the Guinea Pig and Rat

In this example, the toxicity of TCPT was compared between each species. When a female guinea pig received i.v. 10 mg/kg TCPT, there was no observable effect. On the other hand, a female rat receiving i.v. 10 mg/kg was sedated within 30 minutes. In addition, increasing levels of TCPT to i.v. 30 mg/kg in rats resulted in an lethal dose.

The results presented here, although based on two animals per dose group only, give rise to skepticism regarding the degree of analogy of TCPT and TCDD as far as species-differences in acute toxicity is concerned. No amount of TCDD was ever shown to kill animals within five seconds.

### EXAMPLE 7

### High Dose Effect: Altering the Regulation Level for Bodyweight In Adult Female Dunkin-Hartley Guinea Pigs

After a single oral dose of TCDD, rats lose body weight dose-dependently (Seefeld et al. 1984a; Seefeld & Peterson 1984). It can be calculated from the literature (Seefeld et al. 1984b) that once the body burden of TCDD after a non-lethal dose of 15 μg/kg was reduced to 6.6-8 μg/kg (i.e. after 11 days), feed intake started rising again. Below about 5 μg/kg TCDD body burden (3 weeks after dosing), weight gain resumed. TCDD-treated rats then gained weight at the same rate as the controls, although at a lower level. The set-point for bodyweight remained reduced throughout the following observation period (10 weeks), while efficiency of feed utilization was unaffected (Seefeld et al. 1984a).

In the present invention, guinea pigs were dosed for 9 consecutive days with 2.5, 5, 10 and 20 mg/kg/day TCPT, respectively. The observations presented here were made during another study which was not designed for comparative body weight analysis. Hence, only one pair-fed (to 20 mg/kg/day) animal was used. All animals showed a decrease in bodyweight during the dosing period and all but one resumed gaining weight once TCPT administration was terminated. The animal treated with 10 mg/kg/day did not recover and died on day 13. In contrast to studies on TCDD, weight gain resumed 1-2 days after cessation, probably due to TCPT's shorter half-life and therefore more rapid elimination. All survivors showed a reduced set-point of bodyweight, as depicted in comparison of the treated guinea pig with the respective pair-fed animal, and the control animal in FIG. 2.

The set-point for bodyweight was reduced by 66 grams in the treated animal and 79 grams in the pair-fed control. The slope of weight gain remained the same for the pair-fed control but increased in the treated animal after cessation of exposure to TCPT.

### EXAMPLE 8

### Medium Dose Effect: Effects on Ovulation in Adult Dunkin-Hartley Guinea Pigs

In adult cycling rats, a dose of 10 μg/kg TCDD resulted in a prolonged diestrus stage, reduced ovulatory rate and fewer ova shed (Li et al. 1995a). Studies in the immature rat model determined the ED₅₀ for these effects to be between 3 and 10 μg/kg TCDD. Pair-fed controls did not show effects on ovulation, and therefore, an effect of feed intake can be excluded (Li et al. 1995b).

In the present invention, guinea pigs were chosen for this study for two reasons. First, they represent the species that is the most sensitive to TCDD. Second, oral administration was only possible as undissolved solid in gelatin capsules due to the low solubility of TCPT in any solvent. Dosing with gelatin capsules can be performed in this species without anesthesia, which is not possible in rats. This is an important factor, since anesthetics generally influence ovulation.

The guinea pigs were followed for at least two regular consecutive cycles (about 16 days each) before commencing with dosing and also after the administration of TCPT was terminated. Their cycles were tracked by observation of vaginal opening and vaginal smears. The day of ovulation, day 1, was defined as a leucocytic smear following a cornified smear. The animals were dosed for 9 consecutive days starting on day 9 of a highly regular normal cycle. This way, the dosing period overlapped with the relevant time-span for ovulation and regulation of the cycle length (Terranova & Greenwald 1981). The animals received dose rates of 2.5, 5, 10 and 20 mg/kg TCPT per day. One animal was pair-fed to an animal dosed with 20 mg/kg/day TCPT. The pair-fed animal showed a regular cycle length with irregular vaginal opening. Animals in the two lowest dose groups showed no effect, 10 mg/kg/day TCPT resulted in a shortened period of vaginal opening and lethality five days after cessation in one guinea pig. A dose rate of 20 mg/kg/day TCPT inhibited ovulation and led to irregular vaginal opening.

Based on this experiment, it appears TCPT exerts no direct or endocrine effect on the ovaries in guinea pigs, but that the effects on ovulation were merely a result of altered energy regulation in the animals.

### EXAMPLE 9

### Low-Dose Effect: Quantitative In Vitro Ethoxyresorufin-O-Deethylase (“EROD”) Study of TCPT in Rat Hepatoma Cells

The induction of CYP 1A1 has been quantitatively measured in an cell-line study. The choice of EROD as an in vitro study was made based on its established procedure and speed. This revealed the relative AhR interaction of TCPT compared to TCDD as a measure to quantify its dioxin-like induction of gene expression in a cell-based assay. Its metabolic context is both, a strength and a weakness of the EROD assay. The greatest advantage of cell-based assays is the detection of the actual gene expression as compared to the study of interaction with DREs (Seidel et al. 2000).

The EROD-specific activity of TCPT declined over time, with its maximum at 24 h out of measurements after 24, 48 and 72 h. This indicates a comparatively rapid metabilization of TCPT by rat hepatoma cells.

At 24 h, TCPT showed a potency for the induction of in vitro EROD-specific activity of 10^{-2.6} (0.25%) of that of TCDD, as approximately also found in the comparison of the acute i.v. toxicity of both compounds. The efficacy of TCPT reached 77% of the efficacy of TCDD in the in vitro EROD-bioassay. Further studies are currently under investigation for a most accurate determination of the differences in potency and efficacy.

### EXAMPLE 10

### Observed Interaction with Anesthetics

As observed during other experiments, TCPT treatment enhanced the effects of anesthetics. The i.v. administration of 5 mg/kg TCPT in acetone (1 ml/kg) resulted in immediate respiratory arrest in animals that were anesthetized by 55 mg/kg of pentobarbital. CPR was successful in one case; however only for 15 minutes. Animals receiving a 3 mg/kg i.v. dose showed less of an effect, with one animal dying out of three. Peroral administration of solid TCPT 30 min prior to pentobarbital administration (55 mg/kg) shortened the onset of anesthesia by about 50% but lead to no fatalities.

### EXAMPLE 11

### Half-Lives Determined in Rats and Guinea Pigs

Studies on a limited number of animal have been conducted to determine the approximate half-life of TCPT. The kinetics were found to follow a two-compartment model after intravenous dosage with a distribution- and an elimination-phase.

For the distribution phase after intravenous dosing, a half-life below 1 h was found, i.e. 0.82 h, 0.47 h and 0.42 h in two rats dosed 5 mg/kg, one rat dosed 10 mg/kg and two guinea pigs dosed 10 mg/kg, respectively.

The elimination phase was found to be 5.42 h, 2.25 h and 2.71 h in two rats dosed 5 mg/kg, one rat dosed 10 mg/kg and two guinea pigs dosed 10 mg/kg, respectively.

### CONCLUSIONS

Those skilled in the art will appreciate that striking similarities between TCPT and TCDD were found in their effects on bodyweight. TCPT administrated daily exerted a wasting effect analogous to the wasting syndrome known from single dose rates of dioxins. However, as soon as the exposition ceased, animals resumed gaining weight (except for one guinea pig). This is understandable in that the half-life of TCDD is much longer than that of TCPT (weeks vs. hours). Therefore, efficacy is lost more rapidly with the latter. Subsequent weight gain occurred on a reduced scale, with both compounds reducing the set-point for body weight.

The foregoing examples also show several differences between TCPT and TCDD. Their synthesis consists of fusing two aryl rings via heterocyclic bridges. TCPT lacks the energetically advantageous formation-profile of TCDD, and is therefore very difficult to synthesize. Comparing their acute toxicity in animals as well as their in vitro EROD-specific activity, TCPT is by approximately three orders of magnitude less potent than TCDD. The 10⁻⁴-fold difference in acute toxicity found for oral administration is probably due to hepatic first pass effect and/or differences in absorption. Finally, the large species-differences typical for TCDD's acute toxicity could not be confirmed by these preliminary observations. The endocrine effects of TCDD on ovulation in rats could not be reproduced by TCPT in guinea pigs, however, studies of TCPT in rats yet have to be conducted, as do studies of TCDD in guinea pigs.

### PROPHETIC EXAMPLE 1

### Kinetics Studies of TCPT Binding to the Aryl Hydrocarbon Receptor (AhR)

Cytochrome P450s play a major role in phase I metabolism, representing the oxidative enzymes. Compounds can exert transcriptional influence over these proteins via promoters. As first described in 1976 (Poland et al. 1976), TCDD induces CYP 1A1 by interacting with its gene through a receptor-mediated mechanism. The cytoplasmic AhR and its nuclear partner arnt (AhR nuclear translocator) play key roles in this signal transduction. The non-TCDD specific receptor elicits the signaling pathway. An AhR ligand, such as TCDD, enters the cell and interacts with AhR in the cytoplasm. Proteins bound to AhR dissociate to facilitate the binding of TCDD or another substrate to the ligand-binding site. The newly formed receptor/ligand complex migrates into the nucleus and forms a heterotrimer with arnt. This trimer binds to a core heptanucleotide sequence, the dioxin response elements (DREs), in the DNA and initiates transcription. After processing and migration into the cytoplasm, translation of the mRNA takes place and the newly formed proteins elicit a biological response. One manifestation is an increased CYP 1A1 activity in the organism or isolated cell line.

To elucidate the interactions of TCPT with the AhR, receptor-binding studies will be performed. No ³H-TCPT is available, yet, and no radiolabeling of TCPT has been attempted, thus far. Due to this uncertain situation of the availability of labeled compound, studies cannot be proposed based merely on experimental conditions for classical approaches in binding studies. Under these circumstances, there is a wide spectrum of assays for dioxins and dioxin-like compounds (Behnisch et al. 2001) and the Ah-immunoassay (AhIA) (Wheelock et al. 1996) seems to fit the requirements best. It is a commercially available kit (U.S. Pat. No. 6,127,136; Japan Patent No. 3144689), which shows important advantages over the classical approach. However, classical binding studies are outlined below as backup to provide limited validation of the chosen alternative.

Ah-Immunoassay (AhIA)

The Ah-immunoassay is a hybrid of an immunoassay and an in vitro AhR-based assay. Ligands are added to this cell-free test and if they bind to the AhR, the known complex with arnt is formed subsequently. This heterotrimer binds to DREs as in the regular signaling pathway, however, the assay is performed in a cell-free system on a special ELISA plate. After washing, antibodies are added which bind arnt in the complex with AhR, still bound to the plate. Quantification is conducted photometrically. The limit of detection for this assay is given as 1 pg TCDD per sample.

Binding studies of TCPT in the AhIA will be performed with different concentrations of TCDD as a positive control, and the results will be used as standards for quantifying the effects of TCPT. For the study of competitiveness, combinations of both compounds will be investigated.

Instead of the exact quantification of classical binding assays with radiolabeled ligand, the AhIA uses a calibration with TCDD as standard. Concerns may arise from the question if TCDD quantitatively binds in the signaling pathway, since it is used for calibration. The detected activity of TCPT is therefore proportional to the percentage of TCDD bound. This question, however, can be negated by calibrating the test with the low-concentration stretch of first-order TCDD binding kinetics, where quantitative binding applies.

Classical ³H-TCPT Binding Study

Based on the availability of ³H-TCPT, classical binding and displacement studies of TCPT in an AhR assay are planned. The limitations for biological interpretability mentioned earlier are considerable and have to be taken into account. However, binding studies with labeled ligands represent a highly reliable, established system for isolated receptor interactions.

The binding kinetics of ³H-TCPT will be studied. Also, the displacement of ³H-TCPT by TCPT is of interest for the determination of the dynamics, i.e. reversibility, of binding. The displacement of radiolabeled TCDD by TCPT and radiolabeled TCPT by TCDD will provide information about the strength of the ligand interaction with the AhR.

Hypothesis

Based on the structural similarity between TCDD and TCPT, the binding of these two compounds to the AhR is hypothesized to be competitive with agonistic effects. However, due to their differential stereochemistry, a lower affinity of the angled TCPT to the AhR as compared to TCDD is expected. Both compounds are expected to interact with the same ligand-binding domain of the AhR. Consequently, increasing competitive inhibition of the effects of TCDD (AhIA) or displacement of TCDD (classical binding assay) is expected with an increasing TCPT concentration.

### PROPHETIC EXAMPLE 2

### In Silico TCPT/AhR Fitting Studies

The recent solution of the crystal structure of TCPT provides important information for the computational modeling of TCPT/AhR interactions. Although the crystal structure of the AhR has not been determined, yet, in silico studies can be undertaken: Just recently, theoretical research on the structure of the AhR has yielded a three dimensional structure of the ligand-binding domain by homology modeling (Jacobs et al. 2003). Therefore, in addition to the binding studies, calculations will be used to support the investigations of the interactions of TCPT with the AhR.

Hypothesis

A lower but existing goodness-of-fit is hypothesized for TCPT as compared to TCDD, as reasoned for the kinetics studies.

### PROPHETIC EXAMPLE 3

### High-Dose Effect: Inhibition of Phosphoenolpyruvate Carboxykinase (PEPCK) Activity in Adult Female Sprague Dawley Rats

To further study the mechanism of the TCPT-induced loss of bodyweight, as well as to further dissect the mechanism of TCDD-toxicity stated in Chapter 1.1.4, PEPCK inhibition should be measured.

Hypothesis

A dose-dependent inhibition of PEPCK-activity in the same dose-range as toxicity occurs is expected in analogy to TCDD (Rozman, 1992)

### PROPHETIC EXAMPLE 4

### Study of TCPT-Derivatives: p.o. LD₅₀ in Adult Female Sprague-Dawley Rats

Unlike TCDD with its chemically inert structure of two bivalent oxygen atoms in the central ring, TCPT possesses a trivalent nitrogen atom and a sulfur atom that can be bi-, tri- or tetravalent. This offers the possibility of derivatives with properties slightly differing from TCPT. The nitrogen atom can bind groups other than hydrogen such as lower alkyl groups. Further, the sulfur atom is subject to oxidation. The derivatives of primary interest are:

Sulfoxo-Derivatives
TCPT O (2,3,7,8-Tetrachlorophenothiazine-5-oxide)
TCPT O₂ (2,3,7,8-Tetrachlorophenothiazine-5,5-dioxide)

N-Methylated Derivative
N-Me TCPT (N-Methyl-2,3,7,8-Tetrachlorophenothiazine)

Combined Derivatization
N-Me TCPT O (N-Methyl-2,3,7,8-Tetrachlorophenothiazine-5-oxide)
N-Me TCPT O₂ (N-Methyl-2,3,7,8-Tetrachlorophenothiazine-5,5-dioxide)

The acute toxicity of all compounds will be determined for comparison. If no lethality is observed at doses limited by the logistics of administration, such as dosing volume and regimen, the effects of a high, single oral dose on body weight, feed intake and spillage will be determined.

Sulfoxo-Derivatives

In the organism, TCPT is subject to oxidation by cytochrome P450s. As research from many current drugs shows, often metabolites show activity. Since TCPT O and TCPT O₂ are presumed metabolites, a study of their potency as compared to TCPT will be performed. A higher potency of the sulfoxides, however, is not expected, since the oxo-derivatives deviate even more from planarity and therefore from dioxin-like stereochemistry than TCPT does. This would negatively effect their receptor-binding ability.

N-Alkyl Derivatives

TCPT is readily subject to oxidation not only on the sulfur atom, but also on the amine. Formation of the hydroxylamine leads to higher water solubility and, especially after phase II biotransformation, to faster excretion. To slow down the metabolism of TCPT, a lower alkyl group, is used as a protecting group on the amine. Preferably, the lower alkyl group is a methyl group. A higher efficacy of N-Me TCPT as compared to TCPT is expected due to its greater persistence and, if dosing is repeated within four half-lives, its accumulation. If oxidative demethylation does not turn out to be a rate-limiting step in the elimination of N-Me TCPT, then it will behave as TCPT itself.

Among other things, the compounds of the present invention are useful for the treatment of diabetes mellitus (type I and type II), ovulation inhibitor (contraceptive), cancer chemotherapeutic agent, anti-obesity drug (body weight regulator), and immunostimulant.

### PROPHETIC EXAMPLE 6

### Formulations

The compounds of the present invention may be administered in any suitable fashion. For example, the compounds may be administered both orally and parenterally in the dosage form of tablets, powders, granules, capsules, syrups, troches, inhalants, suppositories, injections, ointments, ophthalmic ointments, eye drops, nasal drops, ear drops, cataplasmas, and lotions.

The administration dose widely varies depending on the type of the treatment, the severity of the symptoms, the age, sex and drug sensitivity of the animal. In general, the compounds of the present invention are administered in a daily dose of from about 0.5 mg/kg to 20 mg/kg, preferably from about 3 mg/kg to 15 mg/kg, and most preferably about 5 mg/kg to 10 mg/kg.

The compounds of the present invention may be processed into preparations by conventional methods with the use of conventional pharmaceutical carriers. For example, solid preparations for oral administration are prepared by mixing the principal agent with fillers, binders, disintegrating agents, lubricants, coloring agents, corrigents, antioxidants, etc. and then processed into tablets, coated tablets, granules, powders, capsules, etc. by conventional methods. Examples of the above-mentioned fillers are lactose, corn starch, sucrose, glucose, sorbitol, microcrystalline cellulose, silicon dioxide, etc. Examples of the binders are polyvinyl alcohol, polyvinyl ether, ethylcellulose, methylcellulose, acacia, tragacanth, gelatin, shellac, hydroxypropylcellulose, hydroxypropylmethylcellulose, calcium citrate, dextrin and pectin. Examples of the lubricants are magnesium stearate, talc, polyethylene glycol, silica, hardened vegetable oils, etc. The coloring agents are those admitted to be added to medicines. Examples of the corrigents include cocoa powder, menthol, aromatic powder, peppermint oil, borneol and powdered cinnamon bark. As the antioxidants, use can be made of any pharmaceutically authorized ones such as ascorbic acid and alpha-tocopherol. Tablets and granules may be appropriately coated with sugar, gelatin, and extended release coatings, if necessary. The compounds of the present invention may also be soluble in sodium bicarbonate, thus providing for a suitable injection route.

Since many possible embodiments may be made of the invention without departing from the scope thereof, is to be understood that all matters herein set forth or shown in the accompanying drawings are to be interpreted as illustrative, and not in a limiting sense.

### REFERENCES

Abel, J.: 2,3,7,8-TCDD-Intoxikation beim Menschen. Dioxin-Eine technische, analytische, ökologische und toxikologische Herausforderung. Mannheim, 1987, pp. 487-501.
Allen, J. R., J. P. VanMiller & D. H. Norback: Tissue distribution, excretion and biological effects of [₁₄C]tetrachlorodibenzo-p-dioxin TCDD in rats. Food Cosmet Toxicol 1975, 13, 501.
Angelo-Khattar, M., L. Auger, O. Thulesius & K. Bhatt: Toxicological screening: A three year experience in poisonings in Kuwait. Journal of Toxicology—Clinical Toxicology 1990, 28, 357-364.
Ashe, W. & R. R. Suskind: Chloracne cases of the Monsanto Chemical Company, Nitro, West Virginia. In: Reports of the Kettering Laboratory. The University of Cincinnati, Cincinnati, Ohio, October 1949, April 1950, July 1953.
Atamna, H., M. Krugliak, G. Shalmiev, E. Deharo, G. Pescarmona & H. Ginsburg: Mode of antimalarial effect of methylene blue and some of its analogues on Plasmodium falciparum in culture and their inhibition of P. vinckei petteri and P. yoelii nigeriensis in vivo. Biochem Pharmacol 1996, 51, 693-700.
Beaumont, G., S. Dobbins, D. Latta & W. P. McMillin: Mequitazine in the treatment of hayfever. British Journal of Clinical Practice 1990, 44, 183-188.
Behnisch, P. A., K. Hosoe & S.-i. Sakai: Bioanalytical screening methods for dioxins and dioxin-like compounds—a review of bioassay/biomarker technology. Environment International 2001, 27, 413-439.
Birnbaum, L. S., M. M. McDonald, P. C. Blair, A. M. Clark & M. W. Harris: Differential toxicity of 2,3,7,8-tetrachlorodibenzo-p-dioxin (TCDD) in C57BL/6J mice congenic at the Ah locus. Fundam Appl Toxicol 1990, 15, 186-200.
Blower, P. J. & N.J. Carter: Rapid preparation of 1231-labelled methylene blue and toluidine blue: Potential new agents for parathyroid scintigraphy. Nuclear Medicine Communications 1990, 11, 413-420.
Boer, F. P., F. P. Van Remoortere, P. P. North & M. A. Neuman: The crystal and molecular structure of 2,3,7,8-tetrachlorodibenzo-p-dioxin. Acta Crystallographica 1972, B28, 1023-1029.
Boos, K.-S. & C.-H. Grimm: High-performance liquid chromatography integrated solid-phase extraction in bioanalysis using restricted access precolumn packings. Trends in Analytical Chemistry 1999, 18, 175-180.
Brinton, L. A., J. R. Daling, J. M. Liff, J. B. Schoenberg, K. E. Malone & e. al.: Oral contraceptives and breast cancer risk among younger women. J Natl Cancer Inst 1995, 87, 827-835.
Bunge, M., L. Adrian, A. Kraus, M. Opel, W. G. Lorenz, J. R. Andreesen, H. Görisch & U. Lechner: Reductive dehalogenation of chlorinated dioxins by an anaerobic bacterium. Nature 2003, 421, 357-360.
Carlstedt-Duke, J. M. B.: Tissue distribution of the receptor for 2,3,7,8-tetrachlorodibenzo-p-dioxin in the rat. Cancer Research 1979, 39, 3172-3178.
Choo, H. Y., Y. O. Shin & J. Park: Study of the metabolism of phenothiazines: Determination of N-demethylated phenothiazines in urine. Journal of Analytical Toxicology 1990, 14, 116-119.
Davis, J. W., A. D. Burdick, F. T. Lauer & S. W. Burchiel: The aryl hydrocarbon receptor antagonist, 3′methoxy-4′nitroflavone, attenuates 2,3,7,8-tetrachlorodibenzo-p-dioxin-dependent regulation of growth factor signaling and apoptosis in the MCF-10A cell line. Toxicology and Applied Pharmacology 2003, 188, 42-49.
Dertinger, S. D., H. B. M. Lantum, A. E. Silverstone & T. A. Gasiewicz: Effect of 3′-methoxy-4′-nitroflavone on benzo[a]pyrene toxicity. Aryl hydrocarbon receptor-dependent and -independent mechanisms. Biochemical Pharmacology 2000, 60, 189-196.
DeVito, M. J., L. S. Birnbaum, W. H. Farland & T. A. Gasiewicz: Comparisons of estimated human body burdens of dioxinlike chemicals and TCDD body burdens in experimentally exposed animals. Environ. Health Perspect. 1995, 103, 820-831.
Du-xin Li, Y. Qiao-lian, C. Liang: Synthesis of N-ethylphenothiazine and its derivatives. J. Shanxi Univ., China. 1988, 3, 66-71
Eisler, R.: Dioxin hazards to fish, wildlife, and invertebrates: A synoptic review. Ed.: U. F. a. W. S. B. Report, 1986, pp. 1-38.
Fan, F. & K. K. Rozman: Relationship between acute toxicity of 2,3,7,8-tetrachlorodibenzo.p.dioxin (TCDD) and disturbance of intermadiary metabolism in Long-Evans rats. Archives of Toxicology 1994, 69, 73-78.
Fan, F., D. Wierda & K. K. Rozman: Effects of 2,3,7,8-tetrachlorodibenzo-p-dioxine on humoral and cell-mediated immunity in Sprague-Dawley rats. Toxicology 1996, 106, 221-228.
Fauchier, J. P., D. Babuty, P. Rouesnel, P. Cosnay & J. Estepo: Comparison of the efficacy of moricizine and disopyramide in the treatment of ventricular extrasystoles. Arch Mal Coeur Vaiss 1991, 84, 553-60.
Fernandez-Salguero, P., T. Pineau, M. Hilbert, T. McPhail, S. S. T. Lee, S. Kimura, D. W. Nebert, S. Rudifkoff, M. Ward & F. J. Gonzales: Immune system impairment and hepatic fibrosis in mice lacking the dioxin-binding Ah receptor. Science 1995, 268, 722-726.
Fernandez-Salguero, P. M., D. M. Hilbert, S. Rudikoff, J. M. Ward & F. J. Gonzales: Aryl-hydrocarbon receptor-deficient mice are resistant to 2,3,7,8-tetrachlorodibenzo-p-dion-induced toxicity. Toxicology and Applied Pharmacology 1996, 140, 173-179.
Fingerhut, M. A., W. E. Halpern, D. A. Marlow, L. A. Piacitelli, P. A. Honchar, M. H. Sweeney, A. L. Greife, P. A. Dill, K. Steenland & A. J. Suruda: Cancer mortality in workers exposed to 2,3,7,8-tetrachlorodibenzo-p-dioxin. N Engl J Med 1991, 324, 212-218.
Fowler, G. J., R. C. Rees & R. Devonshire: The photokilling of bladder carcinoma cells in vitro by phenothiazine dyes. Photochemistry and Photobiology 1990, 52, 489-494.
Fried, K. W.: Synthese, Analytik und Aufreinigung von Tetrachlorphenothiazin und Oxoderivaten. In: Fachgebiet Organische Chemie. Ludwig-Maximilians-Universität Munchen, München, 2000.
Gallo, M. A., E. J. Hesse, G. J. MacDonald & T. H. Umbreit: Interactive effects of estradiol and 2,3,7,8-tetrachlorodibenzo-p-dioxin on hepatic cytochrome P-450 and mouse uterus. Toxicol Lett 1986, 32, 123-132.
Gallo, M. J., R. J. Scheuplein & K. A. V. D. Heiden: Biological basis for risk assessment of dioxins and related compounds. In: Barnbury Report. Cold Spring Harbor Laboratory Press, Cold Spring Harbor, N.Y., 1991.
Gasiewicz, T. A.: http://www.urmc.rochester.edu/gebs/faculty/Images/gasfig.jpeg
Gasiewicz, T. A. & R. A. Neal: 2,3,7,8-Tetrachlorodibenzo-p-dioxin tissue distribution, excretion, and effects on clinical chemical parameters in guinea pigs. Toxicol Appl Pharmacol 1979, 51, 329-339.
Geusau, A., K. Abraham, K. Geissler, M. o. Sator, G. Stingl & E. Tschachler: Severe 2,3,7,8-tetrachlorodibenzo-p-dioxin (TCDD) intoxication: Clinical and laboratory effects. Environmental Health Perspectives 2001, 109, 865-869.
Geyer, H. J., K.-W. Schramm, E. A. Feicht, A. Behechti, C. Steinberg, R. Bruggemann, H. Poiger, B. Henkelmann & A. Kettrup: Half-lives of tetra- penta-, hexa-, hepta-, and octachlorodibenzo-p-dioxins in rats, monkeys, and humans—a critical review. Chemosphere 2002, 48, 631-644.
Gocke, E.: Review of genotoxic properties of chlorpromazine and related phenothiazines. Mutation Research 1996, 366, 9-21.
Goodman & Gilman: The pharmacological basis of therapeutics. Eds.: J. G. Hardman, L. E. Limbird and A. G. Gilman. The McGraw-Hill Companies, 2001.
Gorski, J. R., L. W. Weber & K. Rozman: Reduced gluconeogenesis in 2,3,7,8-tetrachlorodibenzo-p-dioxin (TCDD)-treated rats. Arch Toxicol 1990, 64, 66-71.
Graedon, J. & T. Graedon: The People's Pharmacy. St. Martin's Mass Market Paper, 1976.
Gupta, B. N., J. G. Vos, J. A. Moore, J. G. Zinkl & B. C. Bullock: Pathologic effects of 2,3,7,8-tetrachlorodibenzo-p-dioxin in laboratory animals. Environ Health Perspect 1973, 5, 125-140.
Harris, M. W., J. A. Moore, J. G. Vos & B. N. Gupta: General biological effects of TCDD in laboratory animals. Environmental Health Perspectives 1973, 101.
Homberger, E., G. Reggiani, J. Sambeth & H. K. Wipf: The Seveso accident: Its nature, extent and consequences. The Annals of Occupational Hygiene 1979, 22, 327-370.
Houghtaling, M. A., R. Perera, K. E. Owen, S. Wagner, R. J. Kuhn & H. Morrison: Photobiological properties of positively charged methylene violet analogs. Photochem Photobiol 2000, 71, 20-28.
Huff, J.: 2,3,7,8-TCDD: A potent and complete carcinogen in experimental animals. Chemosphere 1992, 25, 173-176.
IÖC: Arbeitsanweisung/SOP Micro-EROD-Assay (in vitro) mit H41IE-Rattenhepatomzellen. GSF —Forschungszentrum für Umwelt und Gesundheit, Neuherberg, 2000.
Jacobs, M. N., M. Dickins & D. F. V. Lewis: Homology modeling of the nuclear receptors: human oestrogen receptor β (hERβ), the human pregnane-X-receptor (PXR), the Ah receptor (AhR) and the constitutive androstane receptor (CAR) ligand binding domains from the human oestrogen receptor a (hERa) crystal structure, and the human peroxisome proliferator activated receptor a(PPARa) ligand binding domain from the human PPAR? crystal structure. Journal of Steroid Biochemistry & Molecular Biology 2003, 84, 117-132.
Jovanovic, M. V. & E. R. Biehl: Correlation of dihedral angles with 13C chemical shifts of quarternary carbon atoms of some phenothiazines and pyrido[3,2-b][1,4]benzothiazine derivatives. J. Heterocyc. Chem. 1987, 24, 51.
Kociba, R. J., D. G. Keyes, J. E. Beyer, R. M. Carreon, C. E. Wade, D. A. Dittenber, R. P. Kaluius, L. E. Franson, C. N. Park, S. D. Bernard, R. A. Hummel & C. G. Humiston: Results of a two-year chronic toxicity and oncogenicity study of 2,3,7,8-tetrachlorodibenzo-p-dioxine in rats. Toxicology and Applied Pharmacology 1978, 46, 279-303.
Koytchev, R., R. G. Alken, V. Kirkov, G. Neshev, M. Vagaday & U. Kunter: Absolute bioavailability of chlorpromazine, promazine and promethazine. Arzneimittelforschung 1994,44,121-125.
Lahaniatis, E. S., E. Clausen, D. Bieniek & F. Korte: Bildung von 2,3,7,8-TCDD bei der Thermolyse von ausgewählten chlorierten organischen Verbindungen. Chemosphere 1985, 14, 233-238.
Lambrecht, B., H. Mohr, J. Knuver Hopf & H. Schmitt: Photoinactivation of viruses in human fresh plasma by phenothiazine dyes in combination with visible light. Vox Sang 1991, 60, 207-13.
Lenoir, D., A. Wehrmeier, K.-W. Schramm, A. Kaune, R. Zimmermann, P. H. Taylor & S. S. Sidhu: Thermal formation of polychlorinated dibenzo-p-dioxins and -furans: Investigations on relevant pathways. Environmental Engineering Science 1998, 15, 37-47.
Li, X., D. C. Johnson & K. K. Rozman: Effects of 2,3,7,8-tetrachlorodibenzo-p-dioxin (TCDD) on estrous cyclicity and ovulation in female Sprague-Dawley rats. Toxicology Letters 1995a, 78, 219-222.
Li, X., D. C. Johnson & K. K. Rozman: Reproductive effects of 2,3,7,8-tetrachlorodibenzo-p-dioxin (TCDD) in female rats: Ovulation, hormonal regulation, and possible mechanism(s). Toxicology and Applied Phamocology 1995b, 133, 312-327.
Lilienfeld, D. E. & M. A. Gallo: 2,4-D, 2,4,5-T, and 2,3,7,8-TCDD: An overview. Epidemiol Rev 1989, 11, 28-58.
Luster, M. I., D. R. Germolec & G. J. Rosenthal: Immunotoxicology—review of current status. Annals of Allergy 1990, 64, 427-432.
Mabey, W. R., J. H. Smith, R. T. Podoll, H. L. Johnson, T. Mill, T. W. Chou, J. Gate, I. Waight-Partridge, H. Jaber & D. Vandenberg: Aquatic Fate Process Data for Organic Priority Pollutants. EPA Report 1982, EPA-440/4-81-014, 107-108.
Mahju, M. A. & R. P. Maickel: Accumulation of phenothiazine tranquilizers in the rat brain and plasma after repeated dosage. Biochem Pharmacol 1969, 18, 2701-2710.
Majors, R. E., K.-S. Boos, C.-H. Grimm, D. Lubda & G. Wieland: Practical guidelines for HPLC-integrated sample preparation using column switching. LC-GC 1996, 14, 554-560.
McKelvey, G. M., E. J. Post, H. E. Jeffery & A. K. Wood: Sedation with promethazine profoundly affects spontaneous airway protection in sleeping neonatal piglets. Clinical and Experimental Pharmacology and Physiology 1999, 26(11), 920-6.
McKinney, J. D., J. Fawkes, S. Jordan, K. Chac, S. Oatley, R. E. Coleman & W. Briner: 2,3,7,8-Tetrachlorodibenzo-p-dioxin (TCDD) as a potent and persistent thyroxine agonist: A mechanistic model based on molecular reactivity. Environ Health Perspect 1985, 61, 41-53.
Meharg, A. A. & K. Killham: A pre-industrial source of dioxins and furans. Nature 2003, 421, 909-910.
Mehtonen, O. P., K. Aranko, L. Malkonen & H. Vapaatalo: A survey of sudden death associated with the use of antipsychotic or antidepressant drugs: 49 cases in Finland. Acta Psychiatr Scand 1991, 84, 58-64.
Miller, J. P. V., J. J. Lalich & J. R. Allen: Increased incidence of neoplasms in rats exposed to low levels of 2,3,7,8-tetrachlorodibenzo-p-dioxin. Chemosphere 1977, 9, 537-544.
Motulsky, H.: http://www.curvefit.com, GraphPad Software, Inc.
Mutzi, G., R. J. Gorski & K. Rozman: Mode of metabolism is altered in 2,3,7,8-tetrachlorodibenzo-p-dioxin (TCDD)-treated rats. Toxicol Lett 1989, 47, 77-86.
Neal, R. A., T. A. Gasiewicz, L. H. Geiger, J. R. Olson & T. Sawahata: Metabolism of 2,3,7,8-tetrachlorodibenzo-p-dioxin in mammalian systems. In: Biological mechanisms of dioxin action. Eds.: A. Poland and R. D. Kimbrough. Cold Spring Harbor Laboratory Press, Cold Spring Harbor, N.Y., 1984, pp. 49.
Neubert, D. & R. Meister: Mutagenic and cancinogenic potential and potency of PCDDs and PCDFs. In: Dioxin—Eine technische, analytische, ökologische und toxikologische Herausforderung. VDI Verlag, Düsseldorf, 1987, pp. 443-486.
Nolan, R. J., F. A. Smith & J. G. Hefner: Elimination and tissue distribution of 2,3,7,8-tetrachlorodibenzo-p-dioxin (TCDD) in female guinea pigs following a single oral dose. Toxicol Appl Pharmacol 1979, 48, 162.
Nowak, J. Z., S. Arbilla, S. G. Dahl & S. Z. Langer: Antagonism of presynaptic dopamine receptors by phenothiazine drug metabolites. Life Sciences 1990, 46, 443-451.
Okamoto, Y. & M. Tomonari: Formation pathways from 2,4,5-trichlorophenol (TCP) to polychlorinated dibenzo-p-dioxins (PCDDs): An ab initio study. J. Chem. Phys. 1999, 103, 7686-7691.
Ott, M. G. & A. Zober: Cause specific mortality and cancer incidence among employees exposed to 2,3,7,8-TCDD after a 1953 reactor incident. Occupational and Environmental Medicine 1996, 53, 606-612.
Pandey, V. K. & S. R. Pathak: Synthesis of 1-(P-arylidenophenyl)-2-oxo-4-methylpyrido [b]phenothiazines. Indian J Chem Sect B 1999, 38, 1111-1113.
Parshikov, I. A., J. P. Freeman, A. J. Williams, J. D. Moody & J. B. Sutherland: Biotransformation of N-acetylphenothiazine by fungi. Appl. Microbiol. Biotechnol. 1999, 52(4), 553-7.
Petrescu, I., O. Bojan, M. Saied, O. Bârzu, F. Schmidt & H. F. Kühnle: Determination of phosphoenylpyruvate carboxykinase acitivity with deoxyguanosine 5′-diphosphate as nucloetide substrate. Analytical Biochemistry 1979, 96, 279-281.
Piper, W. N., J. Q. Rose & P. J. Gehring: Excretion and tissue distribution of 2,3,7,8-tetrachlorodobenzo-p-dioxin in the rat. Environ Health Perspect 1973, 5, 241.
Podoll, R. T., H. M. Jaser & T. Mill: Tetrachlorodibenzo-p-dioxin: Rates of votalization and photolysis in the environment. Environ Sci Technol 1986, 20, 490-492.
Pohjanvirta, R., M. Unkila & J. Tuomisto: Comparative acute lethality of 2,3,7,8-tetrachlorodibenzo-p-dioxin (TCDD), 1,2,3,7,8-pentachlorodibenzo-p-dioxin and 1,2,3,4,7,8-hexachlorodibenzo-p-dioxin in the most TCDD-susceptible and the most TCDD-resistant rat strain. Pharmacol Toxicol 1993, 73, 52-56.
Pohjanvirta, R., T. Vartianinen, A. Uusi-rauva, J. Monkkonen & J. Tuomisto: Tissue distribution, metabolism, and excretion of carbon-14-TCDD in a TCDD-susceptible and a TCDD-resistant rat strain. Pharmacol Toxicol 1990, 66, 93-100.
Poiger, H. & H. R. Buser: The metabolism of TCDD in the dog and rat. In: Biological mechanisms of dioxin action. Eds.: A. Poland and R. D. Kimbrough. Cold Spring Harbor Laboratory Press, Cold Spring Harbor, N.Y., 1984, pp. 39.
Poiger, H., H. R. Buser, H. Weber, U. Zweifel & C. Schlatter: Structure elucidation of mammalian TCDD-metabolites. Experientia 1982, 38, 484-486.
Poiger, H. & C. Schlatter: Pharmacokinetics of 2,3,7,8-TCDD in man. Chemosphere 1989, 15, 1489.
Poland, A., E. Glover & A. S. Kende: Stereospecific, high affinity binding of 2,3,7,8-tetrachlorodibenzo-p-dioxin by hepatic cytosol. Evidence that the binding species is receptor for induction of aryl hydrocarbon hydroxylase. Journal of Biol Chem 1976, 251, 4936-4946.
Poland, A. & J. C. Knutson: 2,3,7,8-Tetrachlorodibenzo-p-dioxin and related halogenated aromatic hydrocarbons: Examination of the mechanism of toxicity. Ann Rev Pharmacol Toxicol 1982, 22, 517-554.
Program, N. T.: Carcinogenesis bioassay of 2,3,7,8-tetrachlorodibenzo-p-dioxin in Osborne-Mendel rats and 36C3F1 mice (gavage study). In: Tech. Rept. Series. National Toxicology Program, Research Triangle Park, NC, 1982, pp. 195.
Rappe, C., H. R. Buser & H.-P. Bosshardt: Dioxins, dibenzofurans and other polyhalogenated aromatics: Production, use, formation and destruction. Ann NY Acad Sci 1979, 320, 1-18.
Romkes, N., J. Piskorska-Ploszczynska & S. Safe: Effects of 2,3,7,8-tetrachlorodibenzo-p-dioxin on hepatic and uterine estrogen receptor levels in rats. Toxicol Appl Pharmacol 1989, 87, 306-314.
Rose, J. Q., J. C. Ramsey, T. H. Wentzler, R. A. Hummel & P. J. Gehring: The fate of 2,3,7,8-tetrachlorodibenzo-p-dioxin following single and repeated oral doses to the rat. Toxicol Appl Pharmacol 1976, 36, 209.
Rozman, K., W. L. Roth, H. Greim, B. U. Stahl & J. Doull: Relative potency of chlorinated dibenzo-p-dioxins (CDDs) in acute, subchronic and chronic (carcinogenicity) toxicity studies: Implications for risk assessment of chemical mixtures. Toxicology 1993, 77, 39-50.
Rozman, K. K.: A critical view on the mechanism(s) of toxicity of 2,3,7,8-tetrachlorodibenzo-p-dioxin. Implications for human safety assessment. Derm Beruf Umwelt 1989, 37, 81-92.
Rozman, K. K.: Search for the mechanism of toxicity of dioxins. Exp Toxic Pathol 1992, 44, 473-480.
Rozman, K. K.: Personal communication, 2003.
Ryan, P. M.: Epidemiology, etiology, diagnosis, and treatment of schizophrenia. Am J Hosp Pharm 1991, 48, 1271-80.
Schmitt, H., H. Mohr, B. Lambrecht & J. Knuver Hopf: Virus inactivation of plasma. Beitr Infusionsther 1991, 28, 82-91.
Schwetz, B. A., J. M. Norris, G. L. Sparschu, U. K. Rowe, P. J. Gehring, J. L. Emerson & C. G. Gerbig: Toxicology of chlorinated dibenzo-p-dioxins. Environmental Health Perspectives 1973, 9, 87-100.
Schlesselman, J. J., B. V. Stadel, M. Korper, W. Yu & P. A. Wingo: Breast cancer detection in relation to oral contraception. J Clin Epidemiol 1992, 45, 1992.
Seefeld, M. D., S. W. Corbett & R. E. Keesey: Characterization of the wasting syndrome in rats treated with 2,3,7,8-tetrachlorodibenzo-p-dioxin. Toxicology and Applied Pharmacology 1984a, 73, 311-322.
Seefeld, M. D., R. E. Keesey & R. E. Peterson: Body weight regulation in rats treated with 2,3,7,8-tetrachlorodibenzo-p-dioxin. Toxicol Appl Pharmacol 1984b, 76, 526-536.
Seefeld, M. D. & R. E. Peterson: Digestible energy and efficiency of feed utilization in rats treated with 2,3,7,8-tetrachlorodibenzo-p-dioxin. Toxicol Appl Pharmacol 1984, 74, 214-222.
Seidel, S. D., V. Li, G. M. Winter, W. J. Rogers, E. I. Martinez & M. S. Denison: Ah Receptor-based chemical screening bioassays: Application and limitations for the detection of Ah receptor agonists. Toxicological Sciences 2000, 55, 107-115.
Sherif, K.: Benefits and risks of oral contraceptives. Am J Obstet Gynecol 1999, 180, 343-348.
Shen, W. W.: A history of antipsychotic drug development. Comprehensive Psychiatry 1999, 40, 407-14.
Simonsen, A. C. & H. Sorensen: Clinical tolerance of methylene blue virus-inactivated plasma—A randomized crossover trial in 12 healthy human volunteers. Vox Sang 1999, 77, 210-217.
Skene, S. A., I. C. Dewhurst & M. Greenberg: Polychlorinated dibenzo-p-dioxins and polychlorinated dibenzofurans: The risk to human health. A review. Hum Toxicol 1989, 8, 173-203.
Stanton, J. M.: Weight gain associated with neuroleptic medication: A review. Schizophr Bull 1995, 21, 463-72.
Stieglitz, L. & H. Vogg: Formation and decomposition of polychlorodibenzo-p-dioxins and -furans in municipal waste incineration. Kernforschungszentrum Karlsruhe, Karlsruhe, 1988.
Stiell, I. G., D. G. Dufour, D. Moher, M. Yen, W. J. Beilby & N. A. Smith: Methotrimeprazine versus meperidine and dimenhydrinate in the treatment of severe migraine: A randomized, controlled trial. Ann Emerg Med 1991, 20, 1201-5.
Terranova, P. F. & G. S. Greenwald: Increased ovulation rate in the cyclic guinea pig after a single injection of antiserum to LH. J Reprod Fert 1981, 61, 37-42.
Thunberg, T.: Effect of TCDD on vitamin A and its relation to TCDD toxicity. In: Biologic mechanisms of dioxin action. Eds.: A. Poland and R. D. Kimbrough. Cold Spring Harbor Laboratory, Cold Spring Harbor, N.Y., 1984, pp. 333-344.
Unkila, M., M. Ruotsalainen, R. Pohjanvirta, M. Viluksela, E. MacDonald, J. T. Tuomisto, K. K. Rozman & J. Tuomisto: Effects of 2,3,7,8-tetrachlorodibenzo-p-dioxin (TCDD) on tryptophan and glucose homeostasis in the most TCDD-susceptible and the most TCDD-resistant species, guinea pigs and hamsters. Archives of Toxicology 1995, 69, 677-683.
Vizir, A. D., Z. E. Grigor'eva, I. V. Stepanova & V. A. Vizir: Membrane-stabilizing preparations in the treatment of patients with heart rhythm disorders of different etiologies. Vrach Delo 1991, 10, 32-6.
Walter, W.: Beyer/Walter. Lehrbuch der Organischen Chemie. In: Beyer/Walter, Lehrbuch der Organischen Chemie. S. Hirzel Verlag, Stuttgart, 1991.
Weber, L. W. D., S. W. Ernst, B. U. Stahl & K. Rozman: Tissue distribution and toxicokinetics of 2,3,7,8-tetrachlorodibenzo-p-dioxin in rats after intravenous injection. Fundamental and Applied Toxcicology 1993, 21, 523-534.
Weber, L. W. D., M. Lebofsky, H. Greim & K. K. Rozman: Key enzymes of gluconeogenesis are dose-dependently reduced in 2,3,7,8-tetrachlorodibenzo-p-dioxin (TCDD)-treated rats. Archives of Toxicology 1991, 65, 119-123.
Weber, L. W. D., M. Lebosky, B. U. Stahl, S. Smith & K. K. Rozman: Correlation between toxicity and effects on intermediary metabolism in 2,3,7,8-tetrachlorodibenzo-p-dioxin-treated male C57BL/6J and DBA/2J mice. Toxicology and Applied Pharmacology 1995, 131, 155-162.
Wheelock, G. D., K. R. Hurst & J. G. Babish: Bioimmunoassay of AhR receptor transformation in vitro by 2,3,7,8-TCDD. Toxicol Methods 1996, 6, 41-50.
Westhoff, C. L.: Breast cancer risk: Perception versus reality. Contraception 1999, 59, 25-28.
WHO: Monographs on the evaluation of the carcinogenic risk of chemicals to man. In: IARC, Geneva, 1977.
Williams & Wilkins: Hazardous Materials Toxicology—Clinical Principles of Environmental Health. Eds.: J. B. Sullivan Jr. and G. R. Krieger, Baltimore, 1992.
Young, A. L.: Determination and measurement of Human Exposure to the Dibenzo-p-Dioxins. Bulletin of Environmental Contamination and Toxicology 1984, 33, 702-709.

1. Phenothiazine compounds having the following formula: 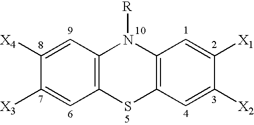
wherein X₁, X₂, X₃, and X₄ are independently hydrogen, halogen, or trihalomethyl, and not more than one of X₁, X₂, X₃, and X₄ is hydrogen; and
wherein R is H or lower alkyl; and
wherein the sulfur is optionally oxidized to sulfoxide or sulfone.

2. The phenothiazine compounds of claim 1 wherein one of X₁, X₂, X₃, and X₄ is halogen; and
wherein at least two of X₁, X₂, X₃, and X₄ are independently trihalomethyl; and
wherein R is H or lower alkyl; and
wherein the sulfur is optionally oxidized.

3. The phenothiazine compounds of claim 1 wherein two of X₁, X₂, X₃, and X₄ are independently halogen; and wherein at least one of X₁, X₂, X₃, and X₄ is independently trihalomethyl; and
wherein R is H or lower alkyl group; and
wherein the sulfur is optionally oxidized.

4. The phenothiazine compounds of claim 1 wherein at least three of X₁, X₂, X₃, and X₄ are independently halogen or trifluoromethyl; and
wherein R is H or lower alkyl; and
wherein the sulfur is optionally oxidized.

5. The phenothiazine compounds of claim 4 wherein three of X₁, X₂, X₃, and X₄, are independently F or trifluoromethyl; and
wherein R is lower alkyl.

6. The phenothiazine compounds of claim 4 wherein at least three of X₁, X₂, X₃ and X₄, are independently Cl or trifluoromethyl; and
wherein R is lower alkyl.

7. The phenothiazine compounds of claim 4 wherein at least three of X₁, X₂, X₃, and X₄ are halogen; and
wherein R is H or lower alkyl; and
wherein the sulfur is optionally oxidized.

8. The phenothiazine compounds of claim 1 wherein X₁, and X₂ are both Cl; and
wherein at least one of X₃, and X₄ is independently halogen or trifluoromethyl; and
wherein R is H or lower alkyl; and
wherein the sulfur is optionally oxidized.

9. The phenothiazine compounds of claim 1 wherein X₁, and X₃ are both Cl; and
wherein at least one of X₂, and X₄ is independently halogen or trifluoromethyl; and
wherein R is H or lower alkyl; and
wherein the sulfur is optionally oxidized.

10. The phenothiazine compounds of claim 1 wherein X₁, and X₄ are both Cl; and
wherein at least one of X₂, and X₃ is independently halogen or trifluoromethyl; and
wherein R is H or lower alkyl; and
wherein the sulfur is optionally oxidized.

11. The phenothiazine compounds of claim 1 wherein X₂, and X₃ are both Cl; and
wherein at least one of X₁, and X₄ are halogen or trifluoromethyl; and
wherein R is H or lower alkyl; and
wherein the sulfur is optionally oxidized.

12. The phenothiazine compounds of claim 1 wherein X₁, and X₂, are both F; and
wherein at least one of X₃, and X₄ is independently halogen or trifluoromethyl; and
wherein R is H or lower alkyl; and
wherein the sulfur is optionally oxidized.

13. The phenothiazine compounds of claim 1 wherein X₁, and X₂ are both I; and
wherein at least one of X₂, and X₄ is independently halogen or trifluoromethyl; and
wherein R is H or lower alkyl; and
wherein the sulfur is optionally oxidized.

14. The phenothiazine compounds of claim 1 wherein X₁, and X₂ are both Br; and
wherein at least one of X₂, and X₄ is independently halogen or trifluoromethyl; and
wherein R is H or lower alkyl; and
wherein the sulfur is optionally oxidized.

15. The phenothiazine compounds of claim 1 wherein X₁ is Cl and X₃ is Br; and
wherein at least one of X₂, and X₄ is independently halogen or trifluoromethyl; and
wherein R is H or lower alkyl; and
wherein the sulfur is optionally oxidized.

16. The phenothiazine compounds of claim 1 wherein X₁, X₂, and X₃, are all Cl; and
wherein at X₄ is hydrogen, halogen, or trifluoromethyl; and
wherein R is H or lower alkyl; and
wherein the sulfur is optionally oxidized.

17. The phenothiazine compounds of claim 16 which is 2,3,7-trichlorophenothiazine.

18. The phenothiazine compound of claim 16 which is 2,3,7-trichlorophenothiazine-5-oxide.

19. The phenothiazine compounds of claim 1 wherein X₁, X₂, and X₄, are all Cl; and
wherein at X₃ is hydrogen, halogen, or trifluoromethyl; and
wherein R is H or lower alkyl; and
wherein the sulfur is optionally oxidized.

20. The phenothiazine compound of claim 19 which is 2,3,8-trichlorophenothiazine.

21. The phenothiazine compound of claim 19 which is 2,3,8-trichlorophenothiazine-5-oxide.

22. The phenothiazine compounds of claim 1 wherein X₁, X₂, and X₃, are all F; and
wherein X₄ is hydrogen, halogen, or trifluoromethyl; and
wherein R is H or lower alkyl; and
wherein the sulfur is optionally oxidized.

23. The phenothiazine compound of claim 22 which is 2,3,7-trifluorophenothiazine.

24. The phenothiazine compound of claim 22 which is N-methyl-2,3,7-trifluorophenothiazine.

25. The phenothiazine compounds of claim 1 wherein X₁, X₂, and X₃, are all Br; and
wherein X₄ is hydrogen, halogen, or trifluoromethyl; and
wherein R is H or lower alkyl; and
wherein the sulfur is optionally oxidized.

26. The phenothiazine compound of claim 25 which is 2,3,7-tribromophenothiazine.

27. The phenothiazine compounds of claim 1 wherein each of X₁, X₂, X₃, and X₄ are independently halogen or trifluoromethyl; and
wherein R is H or lower alkyl; and
wherein the sulfur is optionally oxidized.

28. The phenothiazine compounds of claim 27 wherein R is lower alkyl.

29. The phenothiazine compounds of claim 27 wherein the sulfur is oxidized to a sulfoxide.

30. The phenothiazine compounds of claim 27 wherein the sulfur is oxidized to a sulfone.

31. The phenothiazine compounds of claim 1 wherein X₁, X₂, X₃, and X₄ are all Cl; and
wherein R is H or lower alkyl; and
wherein the sulfur is optionally oxidized.

32. The phenothiazine compound of claim 31 which is 2,3,7,8-tetrachlorophenothiazine.

33. The phenothiazine compound of claim 31 which is 2,3,7,8-tetrachlorophenothiazine-5-oxide.

34. The phenothiazine compound of claim 31 which is 2,3,7,8-tetrachlorophenothiazine-5,5-dioxide.

35. The phenothiazine compound of claim 31 which is N-methyl-2,3,7,8-tetrachlorophenothiazine.

36. The phenothiazine compound of claim 31 which is N-methyl-2,3,7,8-tetrachlorophenothiazine-5-oxide.

37. The phenothiazine compound of claim 31 which is N-methyl-2,3,7,8-tetrachlorophenothiazine-5,5-dioxide.

38. The phenothiazine compounds of claim 1 wherein X₁, X₂, X₃, and X₄ are all F; and
wherein R is H or lower alkyl; and
wherein the sulfur is optionally oxidized.

39. The phenothiazine compound of claim 38 which is 2,3,7,8-tetrafluorophenothiazine.

40. The phenothiazine compound of claim 38 which is 2,3,7,8-tetrafluorophenothiazine-5-oxide.

41. The phenothiazine compound of claim 38 which is 2,3,7,8-tetrafluorophenothiazine-5,5-dioxide.

42. The phenothiazine compound of claim 38 which is N-methyl-2,3,7,8-tetrafluorophenothiazine.

43. The phenothiazine compound of claim 38 which is N-methyl-2,3,7,8-tetrafluorophenothiazine-5-oxide.

44. The phenothiazine compounds of claim 1 wherein X₁, X₂, X₃, and X₄ are all Br; and
wherein R is H or lower alkyl; and
wherein the sulfur is optionally oxidized.

45. The phenothiazine compound of claim 44 which is 2,3,7,8-tetrafluorophenothiazine.

46. The phenothiazine compound of claim 44 which is 2,3,7,8-tetrabromophenothiazine-5-oxide.

47. The phenothiazine compound of claim 44 which is 2,3,7,8-tetrabromophenothiazine-5,5-dioxide.

48. The phenothiazine compound of claim 44 which is N-methyl-2,3,7,8-tetrabromophenothiazine.

49. The phenothiazine compound of claim 44 which is N-methyl-2,3,7,8-tetrabromophenothiazine-5-oxide.

50. The phenothiazine compounds of claim 1 wherein X₁, X₂, X₃, and X₄ are independently Cl or trifluoromethyl; and
wherein R is H or lower alkyl; and
wherein the sulfur is optionally oxidized.

51. The phenothiazine compounds of claim 50 wherein X₁, X₂, X₃, and X₄ are all trifluoromethyl; and
wherein R is H or lower alkyl; and
wherein the sulfur is optionally oxidized.

52. A method of inhibiting ovulation in a mammal comprising administering a therapeutically effective amount of the phenothiazine compound of claim 1 to said mammal.

53. The method of claim 52 further comprising a pharmaceutical carrier for said phenothiazine compound.

54. The method of claim 52 wherein said compound is administered with at least one material selected from the group consisting of a filler, binder, disintegrating agent, lubricants, coloring agent, corrigent, and antioxidant.

55. The method of claim 54 wherein said filler is selected from the group consisting of lactose, corn starch, sucrose, glucose, sorbitol, microcrystalline cellulose, and silicon dioxide.

56. The method of claim 54 wherein said binder is selected from the group consisting of polyvinyl alcohol, polyvinyl ether, ethylcellulose, methylcellulose, acacia, tragacanth, gelatin, shellac, hydroxypropylcellulose, hydroxypropylmethylcellulose, calcium citrate, dextrin and pectin.

57. The method of claim 53 wherein said carrier comprises sodium bicarbonate.

58. The method of claim 52 comprising administering about 0.5 mg/kg/day to 20 mg/kg/day of the phenothiazine compound.

59. The method of claim 52 wherein said mammal is a rat.

60. A method of altering a mammal's body weight comprising administering a therapeutically effective amount of the phenothiazine compound of claim 1 to said mammal.

61. A method of treating type I diabetes comprising administering a therapeutically effective amount of the phenothiazine compound of claim 1 to a mammal.

62. A method of treating type II diabetes comprising administering a therapeutically effective amount of the phenothiazine compound of claim 1 to a mammal.

63. A method of treating and/or preventing cancer in a mammal comprising administering a therapeutically effective amount of the phenothiazine compound of claim 1 to said mammal.

64. A method of treating obesity in a mammal comprising administering a therapeutically effective amount of the phenothiazine compound of claim 1 to said mammal.

65. A method of stimulating the immune system in a mammal comprising administering a therapeutically effective amount of the phenothiazine compound of claim 1 to said mammal.

66. A method of prolonging life in a mammal comprising administering a therapeutically effective amount of the phenothiazine compound of claim 1 to said mammal.
